(19) United States
(12) Patent Application Publication
(10) Pub. No.: US 2024/0309052 A1
(43) Pub. Date: Sep. 19, 2024

(54) CONSTRUCTS, USE AND METHODS THEREOF

(71) Applicant: SUNNYBROOK RESEARCH INSTITUTE, Toronto (CA)

(72) Inventors: Jean Gariepy, Toronto (CA); Shrayasee Saha, Scarborough (CA); Amanda Sparkes, Stouffville (CA)

(21) Appl. No.: 18/606,200

(22) Filed: Mar. 15, 2024

Related U.S. Application Data
(60) Provisional application No. 63/452,617, filed on Mar. 16, 2023.

(51) Int. Cl. C07K 14/005; A61K 39/13; A61P 17/06

(52) U.S. Cl. CPC C07K 14/005; A61K 39/13; A61P 17/06; C07K 2319/30

(57) ABSTRACT

A construct is provided. The construct comprises poliovirus receptor and a Fc domain. In aspects, the PVR is a truncated PVR. Uses of the construct for reducing inflammatory diseases or conditions, reducing symptoms associated with inflammatory diseases or conditions, or reducing immune responses, and associated methods are also provided.

### STATEMENT OF PRIORITY

This application claims the benefit of U.S. Provisional Application Ser. No. 63/452,617, filed Mar. 16, 2023, the entire contents of which are incorporated by reference herein.

### STATEMENT REGARDING ELECTRONIC FILING OF A SEQUENCE LISTING

A Sequence Listing in XML text format, entitled 1360-46_ST26.xml, 18,761 bytes in size, generated on Mar. 12, 2024, and filed herewith, is hereby incorporated by reference into the specification for its disclosures.

### FIELD

The present disclosure relates to constructs. In particular, the present disclosure relates to constructs comprising poliovirus receptor (PVR) and a FC domain, uses and methods thereof.

### BACKGROUND

Poliovirus receptor (PVR; CD155) is a transmembrane glycoprotein involved in forming junctions between neighbouring cells. The extracellular domain (ECD) of the PVR, expressed on antigen presenting cells and tumor cells, for example, is composed of an N-terminal immunoglobulin-like variable (IgV) region followed by two immunoglobulin-like constant (IgC) elements. This ECD has been shown to bind to T-cell immunoglobulin and ITIM domain (TIGIT; also known as WUCAM. VSTM3 and VSIG9), a co-inhibitory receptor that is expressed on activated immune cells, such as CD4⁺ T-cells, CD8⁺ T-cells and NK-cells, where it suppresses their activity (Joller N, et al. Cutting Edge: TIGIT Has T Cell-Intrinsic Inhibitory Functions. The Journal of Immunology 2011;186(3):1338-1342 and Stanietsky N, et al. Mouse TIGIT inhibits NK-cell cytotoxicity upon interaction with PVR. Eur J Immunol 2013;43(8):2138). The inhibition of TIGIT pathways may have implications for cancer immunotherapy, however, the therapeutic potential of engineered products, such as PVR containing constructs, in relation to inflammatory diseases or conditions is relatively unknown.

A need exists for the development of a product, composition and/or method that provides the public with a useful alternative.

The background herein is included solely to explain the context of the disclosure. This is not to be taken as an admission that any of the material referred to was published, known, or part of the common general knowledge as of the priority date.

### SUMMARY

In an aspect, there is a construct comprising poliovirus receptor (PVR) and a Fc domain.

In aspects, the PVR is a truncated PVR.

In aspects, the truncated PVR is free of at least one IgC domain.

In aspects, the truncated PVR is free of both IgC domains.

In aspects, the truncated PVR comprises an immunoglobulin-like variable (IgV) extracellular domain (ECD).

In aspects, the IgV ECD comprises a truncated IgV ECD.

In aspects, the IgV ECD is a human IgV ECD.

In aspects, the IgV ECD is a mouse IgV ECD.

In aspects, the IgV ECD comprises or consists of a polypeptide having at least 80% sequence identity to SEQ ID NO:1:

MAQLARATRS PLSWLLLLFC YALRKAGGDI RVLVPYNSTG
VLGGSTTLHC SLTSNENVTI
TQITWMKKDS GGSHALVAVF HPKKGPNIKE PERVKFLAAQ
QDLRNASLAI SNLSVEDEGI
YECQIATFPR GSRSTNAWLK VQARPKNTAE ALEPSPTLIL
QDVAKCISAN GHPPGRISWP
SNVNGSHREM KEPGSQPGTT TVTSYLSMVP SRQADGKNIT
CTVEHESLQE LDQLLVTLSQ
PYPPENVSIS GYDGNWYVGL TNLTLTCEAH SKPAPDMAGY
NWSTNTGDFP NSVKRQGNML
LISTVEDGLN NTVIVCEVTN ALGSGQGQVH IIVKEKPENM
QQNTRLHLGY IFLIVFVLAV
VIIIAALYTI RRCRHGRALQ SNPSERENVQ YSSVNGDCRL
NMEPNSTR

or a fragment thereof.

In aspects, the IgV ECD comprises or consists of a polypeptide having at least 85, 90, 95, 96, 97, 98, or 99% identity to SEQ ID NO:1, or a fragment thereof.

In aspects, the truncated IgV ECD comprises or consists residues 29 to 147 of SEQ ID NO: 1.

In aspects, the IgV ECD comprises or consists of a polypeptide having at least 80% sequence identity to SEQ ID NO:2:

MARAMAAAWP LLLVALLVLS WPPPGTGDVV VQAPTQVPGF
LGDSVTLPCY LQVPNMEVTH
VSQLTWARHG ESGSMAVFHQ TQGPSYSESK RLEFVAARLG
AELRNASLRM FGLRVEDEGN
YTCLFVTFPQ GSRSVDIWLR VLAKPQNTAE VQKVQLTGEP
VPMARCVSTG GRPPAQITWH
SDLGGMPNTS QVPGFLSGTV TVTSLWILVP SSQVDGKNVT
CKVEHESFEK PQLLTVNLTV
YYPPEVSISG YDNNWYLGQN EATLTCDARS NPEPTGYNWS
TTMGPLPPFA VAQGAQLLIR
PVDKPINTTL ICNVTNALGA RQAELTVQVK EGPPSEHSGI
SRNAIIFLVL GILVFLILLG
IGIYFYWSKC SREVLWHCHL CPSSTEHASA SANGHVSYSA
VSRENSSSQD PQTEGTR

or a fragment thereof.

In aspects, the IgV ECD comprises or consists of a polypeptide having at least 85, 90, 95, 96, 97, 98, or 99% identity to SEQ ID NO:2, or a fragment thereof.

In aspects, the truncated IgV ECD comprises or consists residues 24 to 138 of SEQ ID NO: 2.

In aspects, the Fc domain is an IgG1 Fc domain.

In aspects, the IgG1 Fc domain is a human IgG1 Fc domain.

In aspects, the IgG1 Fc domain is a mouse IgG1 Fc domain.

In aspects, the IgG1 Fc domain comprises or consists of a polypeptide having at least 80% sequence identity to SEQ ID NO:3.

ASTKGPSVFP LAPSSKSTSG GTAALGCLVK DYFPEPVTVS
WNSGALTSGV HTFPAVLQSS
GLYSLSSVVT VPSSSLGTQT YICNVNHKPS NTKVDKKVEP
KSCDKTHTCP PCPAPELLGG
PSVFLFPPKP KDTLMISRTP EVTCVVVDVS HEDPEVKFNW
YVDGVEVHNA KTKPREEQYN
STYRVVSVLT VLHQDWLNGK EYKCKVSNKA LPAPIEKTIS
KAKGQPREPQ VYTLPPSRDE
LTKNQVSLTC LVKGFYPSDI AVEWESNGQP ENNYKTTPPV
LDSDGSFFLY SKLTVDKSRW
QQGNVFSCSV MHEALHNHYT QKSLSLSPGK

or a fragment thereof.

In aspects, the IgG1 Fc domain comprises or consists of a polypeptide having at least 85, 90, 95, 96, 97, 98, or 99% identity to SEQ ID NO:3, or a fragment thereof.

In aspects, the IgG1 Fc domain comprises or consists of residues 100 to 330 of SEQ ID NO:3.

In aspects, the Fc domain is an IgG4 Fc domain.

In aspects, the IgG4 Fc domain is a human IgG4 Fc domain.

In aspects, the IgG4 Fc domain is a mouse IgG4 Fc domain.

In aspects, the IgG4 Fc domain comprises or consists of a polypeptide having at least 80% sequence identity to SEQ ID NO:4.

ASTKGPSVFP LAPCSRSTSE STAALGCLVK DYFPEPVTVS
WNSGALTSGV HTFPAVLQSS
GLYSLSSVVT VPSSSLGTKT YTCNVDHKPS NTKVDKRVES
KYGPPCPSCP APEFLGGPSV
FLFPPKPKDT LMISRTPEVT CVVVDVSQED PEVQFNWYVD
GVEVHNAKTK PREEQFNSTY
RVVSVLTVLH QDWLNGKEYK CKVSNKGLPS SIEKTISKAK
GQPREPQVYT LPPSQEEMTK
NQVSLTCLVK GFYPSDIAVE WESNGQPENN YKTTPPVLDS
DGSFFLYSRL TVDKSRWQEG
NVFSCSVMHE ALHNHYTQKS LSLSLGK

In aspects, the IgG4 Fc domain comprises or consists of a polypeptide having at least 85, 90, 95, 96, 97, 98, or 99% identity to SEQ ID NO:4, or a fragment thereof.

In aspects, the IgG4 Fc domain comprises or consists of residues 99 to 327 of SEQ ID NO:4.

In aspects, the truncated IgV ECD is at the N-terminus of the construct and the Fc domain is at the C-terminus of the construct.

In aspects, the Fc domain is at the N-terminus of the construct and the truncated IgV ECD is at the C-terminus of the construct.

In aspects, the construct further comprises a linker between the PVR and the Fc domain.

In aspects, the linker comprises or consists of the sequence IEGRMD (SEQ ID NO:5).

In aspects, the construct further comprises a secretion signal.

In aspects, the secretion signal comprises or consists of the sequence

(SEQ ID NO: 6)
METDTLLLWVLLLWVPGSTG

In aspects, the construct comprises consists of a polypeptide having at least 80% sequence identity to SEQ ID NO:7:

METDTLLLWVLLLWVPGSTGDIRVLVPYNSTGVLGGSTTLHCSLTSNEN
VTITQITWMKKDSGGSHALVAVFHPKKGPNIKEPERVKFLAAQQDLRNA
SLAISNLSVEDEGIYECQIATFPRGSRSTNAWLKVQARPKNIEGRMDPT
HTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQ
GNVFSCSVMHEALHNHYTQKSLSLSPGK

or a fragment thereof.

In aspects, the construct comprises consists of a polypeptide having at least 85, 90, 95, 96, 97, 98, or 99% identity to SEQ ID NO:7, or a fragment thereof.

In aspects, the construct comprises consists of a polypeptide having SEQ ID NO:7.

In aspects, the construct comprises consists of a polypeptide having at least 80% sequence identity to SEQ ID NO:8:

METDTLLLWVLLLWVPGSTGPGTGDVVVQAPTQVPGFLGDSVTLPCYLQ
VPNMEVTHVSQLTWARHGESGSMAVFHQTQGPSYSESKRLEFVAARLGA
ELRNASLRMFGLRVEDEGNYTCLFVTFPQGSRSVDIWLESKYGPPCPSC
PAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSC
SVMHEALHNHYTQKSLSLSLGK

or a fragment thereof.

In aspects, the construct comprises consists of a polypeptide having at least 85, 90, 95, 96, 97, 98, or 99% identity to SEQ ID NO:8, or a fragment thereof.

In aspects, the construct comprises consists of a polypeptide having SEQ ID NO:8.

In aspects, the construct is soluble.

In aspects, the construct can bind to cognate receptors with similar affinity as compared to a construct comprising a non-truncated PVR.

In aspects, the cognate receptors comprise TIGIT, CD96 and/or CD226.

In aspects, the construct binds to the cognate receptor at low nanomolar affinities, such as, about 0.1 nM to about 10 nM.

In aspects, the construct binds to TIGIT with an affinity of about 2.2 nM.

In aspects, the construct binds to CD96 with an affinity of about 1.7 nM.

In aspects, the construct binds to CD226 with an affinity of about 0.7 nM.

In aspects, the construct suppresses immune responses.

In aspects, the immune response comprises a cytokine response.

In aspects, the cytokine response is IL-2, IFNγ, and/or TNFα.

In aspects, the immune response comprises proliferation of immune cells.

In aspects, the construct suppresses proliferation of immune cells by binding to TIGIT.

In aspects, the immune cells comprise T cells (CD4+ and CD8+) and/NK cells.

In aspects, the construct reduces symptoms associated with a inflammatory disease or condition or for treating an inflammatory disease or condition.

In aspects, the inflammatory disease or condition is selected from Crohn's disease, ulcerative colitis, multiple sclerosis, asthma, rheumatoid arthritis, or psoriasis.

In aspects, the inflammatory disease or condition is Crohn's disease or ulcerative colitis.

In aspects, the symptoms comprise weight loss and/or disease severity.

In aspects, the inflammatory disease or condition is psoriasis.

In aspects, the symptoms comprise erythema, scaling and/or thickness.

In aspects, the construct delays the onset of psoriasis.

In another aspect, there is a polynucleotide encoding the construct described herein.

In aspects, the polynucleotide comprises or consists of a polynucleotide having at least 80% sequence identity to SEQ ID NO:9:

ATGGAAACCGACACACTGCTGCTGTGGGTGCTGCTTCTTTGGGTGCCCG
GAAGCACCGGCGATATCAGAGTGCTGGTGCCCTACAATTCCACCGGCGT
GCTCGGCGGATCTACCACACTGCACTGTAGCCTGACCAGCAACGAGAAC
GTGACCATCACACAGATCACCTGGATGAAGAAGGACAGCGGCGGCTCTC
ATGCTCTGGTGGCCGTGTTTCACCCTAAGAAGGGCCCCAACATCAAAGA
ACCCGAGAGAGTGAAGTTCCTGGCCGCTCAGCAGGACCTGAGAAATGCC
TCTCTGGCCATCAGCAACCTGAGCGTGGAAGATGAGGGCATCTACGAGT
GCCAGATCGCCACCTTTCCTAGAGGCAGCAGAAGCACCAACGCCTGGCT
GAAGGTTCAGGCCAGACCTAAGAACATCGAGGGCAGAATGGACCCCACA
CACACCTGTCCTCCATGTCCTGCTCCAGAGCTGCTCGGAGGCCCTTCCG
TGTTTCTGTTCCCTCCAAAGCCTAAGGACACCCTGATGATCAGCAGAAC
CCCTGAAGTGACCTGCGTGGTGGTGGATGTGTCCCACGAGGATCCCGAA
GTGAAGTTTAATTGGTACGTGGACGGCGTGGAAGTGCACAACGCCAAGA
CCAAGCCTAGAGAGGAACAGTACAACAGCACCTACAGAGTGGTGTCCGT
GCTGACCGTGCTGCACCAGGATTGGCTGAACGGCAAAGAGTACAAGTGC
AAGGTGTCCAACAAGGCCCTGCCTGCTCCTATCGAGAAAACCATCTCCA
AGGCCAAGGGCCAGCCTAGGGAACCCCAGGTTTACACACTGCCTCCAAG
CCGGGAAGAGATGACCAAGAACCAGGTGTCCCTGACCTGCCTGGTCAAG
GGCTTCTACCCTAGCGACATTGCCGTGGAATGGGAGAGCAATGGCCAGC
CTGAGAACAACTACAAGACCACACCTCCTGTGCTGGACAGCGACGGCTC
ATTCTTCCTGTACAGCAAGCTGACAGTGGACAAGAGCAGATGGCAGCAG
GGCAACGTGTTCAGCTGCAGCGTGATGCACGAGGCCCTGCACAACCACT
ACACCCAGAAGTCCCTGAGCCTGTCTCCTGGCAAGTGATGATGAA

or a fragment thereof.

In aspects, the polynucleotide comprises or consists of a polynucleotide having at least 80% sequence identity to SEQ ID NO:10:

AUGGAAACCGACACACUGCUGCUGUGGGUGCUGCUUCUUUGGGUGCCCG
GAAGCACCGGCGAUAUCAGAGUGCUGGUGCCCUACAAUUCCACCGGCGU
GCUCGGCGGAUCUACCACACUGCACUGUAGCCUGACCAGCAACGAGAAC
GUGACCAUCACACAGAUCACCUGGAUGAAGAAGGACAGCGGCGGCUCUC
AUGCUCUGGUGGCCGUGUUUCACCCUAAGAAGGGCCCCAACAUCAAAGA
ACCCGAGAGAGUGAAGUUCCUGGCCGCUCAGCAGGACCUGAGAAAUGCC
UCUCUGGCCAUCAGCAACCUGAGCGUGGAAGAUGAGGGCAUCUACGAGU
GCCAGAUCGCCACCUUUCCUAGAGGCAGCAGAAGCACCAACGCCUGGCU
GAAGGUUCAGGCCAGACCUAAGAACAUCGAGGGCAGAAUGGACCCCACA
CACACCUGUCCUCCAUGUCCUGCUCCAGAGCUGCUCGGAGGCCCUUCCG
UGUUUCUGUUCCCUCCAAAGCCUAAGGACACCCUGAUGAUCAGCAGAAC
CCCUGAAGUGACCUGCGUGGUGGUGGAUGUGUCCCACGAGGAUCCCGAA
GUGAAGUUUAAUUGGUACGUGGACGGCGUGGAAGUGCACAACGCCAAGA
CCAAGCCUAGAGAGGAACAGUACAACAGCACCUACAGAGUGGUGUCCGU
GCUGACCGUGCUGCACCAGGAUUGGCUGAACGGCAAAGAGUACAAGUGC
AAGGUGUCCAACAAGGCCCUGCCUGCUCCUAUCGAGAAAACCAUCUCCA
AGGCCAAGGGCCAGCCUAGGGAACCCCAGGUUUACACACUGCCUCCAAG
CCGGGAAGAGAUGACCAAGAACCAGGUGUCCCUGACCUGCCUGGUCAAG
GGCUUCUACCCUAGCGACAUUGCCGUGGAAUGGGAGAGCAAUGGCCAGC
CUGAGAACAACUACAAGACCACACCUCCUGUGCUGGACAGCGACGGCUC
AUUCUUCCUGUACAGCAAGCUGACAGUGGACAAGAGCAGAUGGCAGCAG
GGCAACGUGUUCAGCUGCAGCGUGAUGCACGAGGCCCUGCACAACCACU
ACACCCAGAAGUCCCUGAGCCUGUCUCCUGGCAAGUGAUGAUGAA

or a fragment thereof.

In aspects, the polynucleotide comprises or consists of a polynucleotide having at least 80% sequence identity to SEQ ID NO:11:

ATGGAAACCGACACACTGCTGCTGTGGGTGCTGCTTCTTTGGGTGCCCG
GATCTACAGGACCTGGAACAGGCGACGTTGTGGTGCAGGCTCCTACTCA
GGTGCCAGGCTTTCTGGGCGATAGCGTGACCCTGCCTTGCTACCTGCAA
GTGCCCAACATGGAAGTGACCCACGTGTCCCAGCTGACCTGGGCTAGAC
ATGGCGAGTCTGGATCCATGGCCGTGTTCCACCAGACACAGGGCCCTAG
CTACAGCGAGAGCAAGCGGCTGGAATTTGTGGCCGCTAGACTGGGAGCC
GAGCTGAGAAATGCCAGCCTGAGAATGTTCGGCCTGAGAGTGGAAGATG
AGGGCAACTACACCTGTCTGTTCGTGACCTTTCCACAGGGCAGCAGAAG
CGTGGACATCTGGCTGGAATCTAAGTACGGCCCTCCTTGTCCTAGCTGC
CCCGCTCCTGAATTTCTCGGCGGACCTTCCGTGTTCCTGTTTCCTCCAA
GGAAGTGCACAACGCCAAGACCAAGCCTAGAGAGGAACAGTTCAACAGC
AAGCCTAAGGACACCCTGATGATCAGCAGAACCCCTGAAGTGACCTGCG
TGGTGGTGGATGTGTCCCAAGAGGATCCCGAGGTGCAGTTCAATTGGTA
CGTGGACGGCGTCCTACAGAGTGGTGTCCGTGCTGACCGTGCTGCACCA
GGATTGGCTGAACGGCAAAGAGTACAAGTGCAAGGTGTCCAACAAGGGC
CTGCCTAGCAGCATCGAGAAAACCATCAGCAAGGCCAAGGGCCAGCCAA
GAGAACCCCAGGTGTACACACTGCCTCCAAGCCAAGAGGAAATGACCAA
GAACCAGGTGTCCCTGACCTGCCTGGTCAAGGGCTTCTACCCTTCCGAT
ATCGCCGTGGAATGGGAGAGCAATGGACAGCCCGAGAACAACTACAAGA
CCACACCTCCTGTGCTGGACAGCGACGGCTCATTCTTCCTGTACAGCAG
ACTGACCGTGGACAAGAGCAGATGGCAAGAGGGCAACGTGTTCAGCTGC
AGCGTGATGCACGAGGCCCTGCACAACCACTACACCCAGAAGTCTCTGA
GCCTGAGCCTGGGCAAGTGATGATGA

or a fragment thereof.

In aspects, the polynucleotide comprises or consists of a polynucleotide having at least 80% sequence identity to SEQ ID NO:12:

AUGGAAACCGACACACUGCUGCUGUGGGUGCUGCUUCUUUGGGUGCCCG
GAUCUACAGGACCUGGAACAGGCGACGUUGUGGUGCAGGCUCCUACUCA
GGUGCCAGGCUUUCUGGGCGAUAGCGUGACCCUGCCUUGCUACCUGCAA
GUGCCCAACAUGGAAGUGACCCACGUGUCCCAGCUGACCUGGGCUAGAC
AUGGCGAGUCUGGAUCCAUGGCCGUGUUCCACCAGACACAGGGCCCUAG
CUACAGCGAGAGCAAGCGGCUGGAAUUUGUGGCCGCUAGACUGGGAGCC
GAGCUGAGAAAUGCCAGCCUGAGAAUGUUCGGCCUGAGAGUGGAAGAUG
AGGGCAACUACACCUGUCUGUUCGUGACCUUUCCACAGGGCAGCAGAAG
GACCUUCCGUGUUCCUGUUUCCUCCAAAGCCUAAGGACACCCUGAUGAU
CCGUGGACAUCUGGCUGGAAUCUAAGUACGGCCCUCCUUGUCCUAGCUG
CCCCGCUCCUGAAUUUCUCGGCGAGCAGAACCCCUGAAGUGACCUGCGU
GGUGGUGGAUGUGUCCCAAGAGGAUCCCGAGGUGCAGUUCAAUUGGUAC
GUGGACGGCGUGGAAGUGCACAACGCCAAGACCAAGCCUAGAGAGGAAC
AGUUCAACAGCACCUACAGAGUGGUGUCCGUGCUGACCGUGCUGCACCA
GGAUUGGCUGAACGGCAAAGAGUACAAGUGCAAGGUGUCCAACAAGGGC
CUGCCUAGCAGCAUCGAGAAAACCAUCAGCAAGGCCAAGGGCCAGCCAA
GAGAACCCCAGGUGUACACACUGCCUCCAAGCCAAGAGGAAAUGACCAA
GAACCAGGUGUCCCUGACCUGCCUGGUCAAGGGCUUCUACCCUUCCGAU
AUCGCCGUGGAAUGGGAGAGCAAUGGACAGCCCGAGAACAACUACAAGA
CCACACCUCCUGUGCUGGACAGCGACGGCUCAUUCUUCCUGUACAGCAG
ACUGACCGUGGACAAGAGCAGAUGGCAAGAGGGCAACGUGUUCAGCUGC
AGCGUGAUGCACGAGGCCCUGCACAACCACUACACCCAGAAGUCUCUGA
GCCUGAGCCUGGGCAAGUGAUGAUGA

or a fragment thereof.

In another aspect, there is a vector comprising the polynucleotide described herein.

In yet another aspect, there is a host cell comprising the vector described herein.

In yet another aspect, there is a composition comprising the construct described herein.

In still another aspect, the construct described herein, the polynucleotide described herein, the vector described herein, the host cell described herein, or the composition described herein are for suppressing an immune response.

In still another aspect, the construct described herein, the polynucleotide described herein, the vector described herein, the host cell described herein, or the composition described herein are for treating and/or preventing an inflammatory condition.

In still another aspect, there is a method for supressing an immune response, the method comprises administering the construct described herein to a subject in need thereof.

In still another aspect, there is a method for treating an inflammatory disease or condition, the method comprising administering the construct described herein to a subject in need thereof.

In still another aspect, there is a method for reducing symptoms associated with an inflammatory disease or condition, the method comprising administering the construct described herein to a subject in need thereof.

In yet still another aspect, there is a use of the construct described herein for delaying onset of psoriasis.

In yet still another aspect, there is a use of the construct described herein for supressing an immune response.

In yet still another aspect, there is a use of the construct of described herein for treating an inflammatory disease or condition.

In yet still another aspect, there is a use of the construct described herein for reducing symptoms associated with an inflammatory disease or condition.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 shows a ribbon structure representation of vdPVR-Fc construct generated using ColabFold; Color pattern: vdPVR (in green), linker (in purple) and Fc domain (in blue).

FIGS. 2A-2C show an example of design and purification of murine vdPVR-Fc and mutant vdPVR-Fc. (A) Diagram depicting the structure of IgV-domain PVR-Fc (vdPVR-Fc) and mutant vdPVR-Fc. The vdPVR-Fc recombinant construct consists of the N-terminal murine IgV portion of the PVR extracellular domain (residues 29-147; Accession #NP_081790.1) connected by a short linker (IEGRMDP) to a human IgG1 Fc domain (residues 100-330; Accession #P01857) located at its C-terminus. Mutant vdPVR-Fc contains two point mutations in the PVR IgV domain (Q62R and F128R). (B) Ribbon structure representation of the mutant vdPVR-Fc construct generated using ColabFold. Color pattern: vdPVR (in green), linker (in purple) and Fc domain (in blue). Q62R and F128R mutations in vdPVR are highlighted in red. (C) The purity and molecular mass of both proteins were confirmed by SDS-PAGE (left panel) in the presence or absence of a reducing agent (DTT). Western blot (right panel) of both constructs in the absence of DTT (blot detected using an anti-human Fc fragment antibody conjugated to HRP). Both expressed proteins migrated as a 94 kDa band in their dimeric form.

FIGS. 3A-3C show surface plasmon resonance (SPR) titration profiles of vdPVR-Fc, mutant vdPVR-Fc and PVR-Fc binding to mouse TIGIT, mouse CD226 and mouse CD96. (A-C) SPR single-cycle kinetics sensorgrams (in red) and fitted curves (in black) with equilibrium association (KD) constant showing the binding of vdPVR-Fc, mutant vdPVR-Fc and full length PVR-Fc to immobilized recombinant (A) mouse TIGIT-Fc-his, (B) mouse CD226-his and (C) CD96-his. Five concentrations (indicated on the sensorgrams) of vdPVR-Fc, mutant vdPVR-Fc and PVR-Fc were flown over immobilized recombinant proteins. Kinetic parameters were calculated from 1:1 Langmuir binding model using Biacore evaluation software.

FIG. 4 shows vdPVR-Fc binds to primary mouse T-cells. Stimulated T-cells in mixed splenocyte cultures were incubated with about 2.5 μg of either vdPVR-Fc and mutant vdPVR-Fc. Histograms depicting vdPVR-Fc (blue) and mutant vdPVR-Fc (blue) binding to CD4⁺ and CD8⁺ T-cells compared with a human IgG1isotype control (red). Binding of vdPVR-Fc and mutant vdPVR-Fc to T-cells was detected with an anti-human IgG1 Fc antibody conjugated to PE. Data is representative of 2 independent experiments.

FIGS. 5A-5D show vdPVR-Fc as a soluble ligand that suppresses T-cell activity in mouse splenocyte cultures ex-vivo. (A, B) Graphs show the mean proliferation percentages of CD4⁺ (A) and CD8⁺ (B) T-cells unstimulated, stimulated with anti-CD3/CD28 coated beads, or with anti-CD3/CD28 beads in the presence of either vdPVR-Fc (about 10 μg/mL) or hIgG (about 10 μg/mL) after 3 days of culture. Proliferation was measured by flow cytometry as a measure of the percentage of CFSE signal associated with cell divisions over 3 days. The culture supernatants were harvested from the murine splenocyte culture after 3 days and the level of (C) IL-2 and (D) IFNγ were quantified by ELISA. Dashed line represents maximum stimulation using anti-CD3/CD28 coated beads. Each point on the graph represents a biological replicate. Error bars represent mean±SEM, * P<0.05, ** P<0.01 and **** P<0.0001 by one-way ANOVA relative to anti-CD3/CD28 beads alone.

FIGS. 6A-6D show vdPVR-Fc as a soluble ligand that suppresses T-cell activity in mouse splenocyte cultures ex-vivo. (A, B) Graphs show the mean proliferation percentages of CD4⁺ (A) and CD8⁺ (B) T-cells unstimulated, stimulated with anti-CD3/CD28 coated beads, or with anti-CD3/CD28 beads in the presence of either vdPVR-Fc (about 10 μg/mL) or mutant vdPVR-Fc (about 10 μg/mL) after 3 days of culture. Proliferation was measured by flow cytometry as a measure of the percentage of CFSE signal associated with cell divisions over 3 days. The culture supernatants were harvested from the murine splenocyte culture after 3 days and the level of (C) IL-2 and (D) IFNγ were quantified by ELISA. Dashed line represents maximum stimulation using anti-CD3/CD28 coated beads. Each point on the graph represents a biological replicate. Error bars represent mean±SEM, * P<0.05, ** P<0.01 and **** P<0.0001 by one-way ANOVA relative to anti-CD3/CD28 beads alone.

FIGS. 7A-7B show expression of PVR cognate receptors on T-cells and cell binding competition. (A) TIGIT, CD226 and CD96 expression on CD3⁺ T-cells was assessed after 1 day of stimulation with anti-CD3/CD28 beads. (B) Flow cytometry histograms showing vdPVR-Fc binding to stimulated CD4⁺ and CD8⁺ T-cell subsets (blue) and vdPVR-Fc binding after the addition of 2-molar excess anti-TIGIT antibody (red). Data are representative of 2 independent experiments.

FIG. 8 shows cytotoxicity of vdPVR-Fc and mutant vdPVR-Fc towards mouse splenocytes. Cytotoxicity was determined by ELISA by measuring the level of lactate dehydrogenase released in cell supernatants. Cytotoxicity (%)=((Test Sample−Low Control)/(High Control−Low Control))×100, where cells cultured in the absence of proteins served as a baseline negative control and signal recorded following complete cell lysis represented the positive control. Each point represents a biological replicate. (n=2; error bars represent mean±SEM). An isotype-matched human IgG1 and an anti-human CTLA mAb served as controls for measuring the cytotoxic effect of the Fc domain present in the constructs on mouse splenocytes.

FIG. 9 shows an experimental outline of a DSS-induced colitis model. Colitis was induced by 2% DSS in the drinking water for 7 days. Mice receiving DSS were treated on days 3 and 5 with either vdPVR-Fc or hIgG isotype control.

FIGS. 10A-10C show treatment with vdPVR-Fc attenuates the symptoms of DSS-induced colitis related weight loss and disease severity in mice. (A) Colitis symptoms for each group (n=4-5) are reported as Disease Activity Index (DAI), a cumulative score of weight loss, stool consistency and rectal bleeding. (B) Percent weight loss relative to Day 3 was monitored. Treatment with vdPVR-Fc significantly reduced disease severity and percent weight loss compared to untreated mice on DSS (adjusted p value, two-way ANOVA). (C) Colon lengths were measured on Day 7 with each point representing a biological replicate. Error bars represents mean±SD, * P<0.05, ** P<0.01 and **** P<0.0001 relative to DSS group evaluated by ANOVA.

FIGS. 11A-11C show vdPVR-Fc delays the onset of psoriatic symptoms in a murine model of imiquimod-induced psoriasis. Female C57BL/6 mice received a daily topical application of either Vaseline or about 5% imiquimod (IMQ) cream on their shaved backs for 5 days. IMQ-treated mice received intraperitoneal administration of either vdPVR-Fc or mutant vdPVR-Fc every other day starting from Day 0. (A) Representative images of mouse back skin on Day 2 after treatment with either Vaseline, imiquimod alone, imiquimod and vdPVR-Fc or imiquimod and mutant vdPVR-Fc. (B) Individual (erythema, scaling, and thickness) and cumulative scores of the treatment groups (n=5). Graphs show an average score of 3 independent scorers comparing each group to IMQ alone (adjusted p value, evaluated by two-way ANOVA). (C) Hematoxylin and eosin staining of back skin sections of Vaseline, imiquimod alone, vdPVR-Fc and mutant vdPVR-Fc treated mice. Back skin samples were collected on Day 3. Epidermal thickness was quantified from histology sections (n=9). Error bars represent mean±SD, * P<0.05, ** P<0.01 and **** P<0.0001 relative to IMQ alone group evaluated by ANOVA.

FIG. 12 shows vdPVR-Fc reduces CD11b⁺ NKp46⁺ NK-cells in the psoriatic skin. Immune cell populations in the back skin samples of mice (n=6-8) were analyzed by flow cytometry on Day 3. Treatment with vdPVR-Fc significantly reduces CD11b⁺ NKp46⁺ NK-cells within the skin compared to mice receiving IMQ alone. Each point represents a biological replicate. Error bars represents mean±SEM, * p<0.05 relative to IMQ alone group evaluated by one-way ANOVA.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

An “immunomodulator” refers to any molecule that can modulate (e.g. change, start, stop, increase, decrease, etc) an immune response. The molecule can be any suitable molecule, such as, proteins, peptides, nucleic acids, amino acids, nucleosides, antibodies, antibody fragments, antibody ligands, peptide nucleic acids, small organic molecules, lipids, hormones, drugs, enzymes, lectin, cell adhesion molecule, antibody epitope, enzyme substrates, enzyme inhibitors, coenzymes, inorganic molecules, carbohydrates, such as polysaccharides and monosaccharides, or a combination thereof. In typical aspects, the molecule is a protein. Examples of immunomodulators are provided in the description below.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Variants” are biologically active constructs, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N-or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

The constructs described herein may include modifications. Such modifications include, but are not limited to, conjugation to an effector molecule. Modifications further include, but are not limited to conjugation to detectable reporter moieties. Modifications that extend half-life (e.g., pegylation) are also included. Proteins and non-protein agents may be conjugated to the constructs by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

“Active” or “activity” for the purposes herein refers to a biological and/or an immunological activity of the constructs described herein, wherein “biological” activity refers to a biological function (either inhibitory or stimulatory) caused by the constructs.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the immunogen, age, sex, and weight of the subject. Dosage or treatment regimes may be adjusted to provide the optimum therapeutic response, as is understood by a skilled person. For example, administration of a therapeutically effective amount of the constructs described herein is, in aspects, sufficient to reduce the proliferation of immune cells (e.g. T cells and/or NK cells). In another example, administration of a therapeutically effective amount of the construct described herein is, in aspects, sufficient to treat and/or prevent an inflammatory disease or condition, or to reduce the symptoms associated therewith.

Moreover, a treatment regime of a subject with a therapeutically effective amount may consist of a single administration, or alternatively comprise a series of applications. The length of the treatment period depends on a variety of factors, such as the immunogen, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The constructs described herein may, in aspects, be administered before, during or after treatment with conventional therapies for the disease or condition in question, such as ulcerative colitis or psoriasis.

The term “condition” indicates, for example, a physical status of a mammal (as a whole or as one or more of its parts), that does not conform to a standard physical status associated with a state of well-being for the mammal. Conditions herein described include but are not limited to disorders and diseases wherein the term “disorder” indicates, for example, a condition of the mammal that is associated to a functional abnormality of the mammal or of any of its parts, and the term “disease” indicates, for example, a condition of the mammal that impairs normal functioning of the body of the mammal or of any of its parts and is typically manifested by distinguishing signs and symptoms.

The term “treating inflammatory disease or condition” or “treatment of inflammatory disease or condition ” refers to administration to a mammal afflicted with a inflammatory disease or condition and refers to an effect that alleviates the inflammatory disease or condition by, for example, reducing symptoms associated therewith, or by targeting cells that contribute to the inflammatory disease or condition pathology.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order. The term “administration” (e.g., “administering” a compound) in reference to a compound, composition and/or formulation disclosed herein includes, for example, introducing the compound, composition and/or formulation into the system of the mammal in need of treatment. When a compound, composition and/or formulation is provided in combination with one or more other active agents, “administration” and its variants are each understood to include concurrent and sequential introduction of the compound, composition and/or formulation and other agents.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration. The term “pharmaceutically acceptable” includes those compounds, materials, compositions, and/or dosage forms which are, within the scope of sound medical judgment, suitable for use in contact with the tissues of humans and animals without excessive toxicity, irritation, allergic response, or other problem or complication commensurate with a reasonable benefit/risk ratio.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

When introducing elements disclosed herein, the articles “a”, “an”, “the”, and “said” are intended to mean that there may be one or more of the elements.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. It will be understood that any embodiments described as “comprising” certain components may also “consist of” or “consist essentially of,” these components, wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects described herein. For example, a composition defined using the phrase “consisting essentially of” encompasses any known acceptable additive, excipient, diluent, carrier, and the like, suitable for the composition described herein. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation, such as any specific compounds or method steps, whether implicitly or explicitly defined herein.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

The phrase “at least one of” is understood to be one or more. The phrase “at least one of . . . and . . . ” is understood to mean at least one of the elements listed or a combination thereof, if not explicitly listed. For example, “at least one of A, B, and C” is understood to mean A alone or B alone or C alone or a combination of A and B or a combination of A and C or a combination of B and C or a combination of A, B, and C.

Described herein, in aspects, are constructs. The construct comprises poliovirus receptor (PVR) and a Fc domain. In aspects, the PVR and the Fc domain are fused to one another, and in other aspects, the PVR and the Fc domain are fused to one another directly, or indirectly. In these aspects, the construct is referred to as a fusion construct.

PVR, also known as CD155 (cluster of differentiation 155), is a transmembrane glycoprotein with an N-terminal signal sequence, three extracellular immunoglobulin (Ig)-like domains, a transmembrane domain, and a cytoplasmic tail. PVR has a molecular size of approximately 80 kDa and a structure consisting of three Ig-like domains (specifically the outermost IgV-like domain followed by two IgC2-like domains; IgV-IgC-IgC).

In aspects, the PVR of the construct is a truncated PVR. In aspects, the truncated PVR is free of at least one immunoglobulin-like constant (IgC) domain, and in other aspects, the truncated PVR is free of two IgC domains. In other words, the truncated PVR described herein may not comprise one IgC domain, or the truncated PVR described herein may not comprise both IgC domains. In other aspects, the truncated PVR comprises an immunoglobulin-like variable (IgV) extracellular domain (ECD). Based on the foregoing, truncated PVR may comprise IgV-IgC, or it may comprise IgV alone. In further aspects, the IgV ECD comprises a truncated IgV ECD. By the use of the term “truncated”, it is understood that the PVR or the IgV ECD is not the length of the full-length sequence. For greater clarity, if at least one amino acid from the full-length sequence is removed, the sequence would be considered be truncated. A truncated sequence can also refers to the specific resides in the sequence, for example, resides 5 to 25 of the sequence, would be considered a truncated sequence. Because the IgV ECD is truncated, in aspects, a mass of the truncated IgV ECD, relative to the full length PVR ECD, is reduced by about 70 kDa.

Surprisingly, while the construct described herein has a truncated PVR or truncated IgV ECD, the construct can still bind to its cognate receptors (also referred to as immunomodulators herein) similarly to a construct comprising a non-truncated PVR. Removal of the IgC domain(s) could render the produced protein unstable and/or with reduced binding affinity, however, the constructs described herein comprising the truncated PVR or truncated IgV ECD bind to their targets with a similar affinity as a construct consisting of the full-length PVR-Fc construct (described in more detail below). Thus, not only can the truncated IgV represent the smallest portion of the ECD which is functional (e.g. has binding capabilities), but it may also offer advantages over a full-length PVR-Fc construct, such as, for example, improved biodistribution in tissues, increased ability/ease of manufacturing, increased tissue penetration and/or enhanced therapeutic efficiency. For example, smaller antibody fragments and recombinant proteins such as single-chain variable fragments (scFv) or diabodies have been previously shown to display superior tumor tissue uptake and distribution while still retaining high affinity for target. Additionally, smaller protein fragments can be readily linked to other targeting domains to generate multivalent agents to modulate different cell populations and/or signalling pathways (Li, Z. et al. Influence of molecular size on tissue distribution of antibody fragments. MAbs 8, 113-119 (2016); Holliger, P., Hudson, P. Engineered antibody fragments and the rise of single domains. Nat Biotechnol 23, 1126-1136 (2005). https://doi.org/10.1038/nbt1142). In view of these examples, the constructs described herein and the proteins produced therefrom may therefore function in a similar way and/or may be manipulated as described above, to, for example, increase biodistribution in tissues, tissue penetration and/or therapeutic efficiency in vivo.

The IgV ECD can be of any mammalian source, such as, from humans, other higher primates, domestic and farm animals, and zoo, sports, or pet animals, such as mice, dogs, cats, cattle, horses, sheep, pigs, goats, or rabbits. In aspects, the IgV ECD is a human IgV ECD, and in other aspects, the IgV ECD is a mouse IgV ECD.

In aspects, the IgV ECD comprises or consists of a polypeptide having at least 80% sequence identity to SEQ ID NO:1:

MAQLARATRS PLSWLLLLFC YALRKAGGDI RVLVPYNSTG
VLGGSTTLHC SLTSNENVTI
TQITWMKKDS GGSHALVAVF HPKKGPNIKE PERVKFLAAQ
QDLRNASLAI SNLSVEDEGI
YECQIATFPR GSRSTNAWLK VQARPKNTAE ALEPSPTLIL
QDVAKCISAN GHPPGRISWP
SNVNGSHREM KEPGSQPGTT TVTSYLSMVP SRQADGKNIT
CTVEHESLQE LDQLLVTLSQ
PYPPENVSIS GYDGNWYVGL TNLTLTCEAH SKPAPDMAGY
NWSTNTGDFP NSVKRQGNML
LISTVEDGLN NTVIVCEVTN ALGSGQGQVH IIVKEKPENM
QQNTRLHLGY IFLIVFVLAV
VIIIAALYTI RRCRHGRALQ SNPSERENVQ YSSVNGDCRL
NMEPNSTR

or a fragment thereof. In other aspects, the IgV ECD comprises or consists of a polypeptide having at least 85, 90, 95, 96, 97, 98, or 99% identity to SEQ ID NO:1, or a fragment thereof. In aspects, the truncated IgV ECD comprises or consists residues 29 to 147 of SEQ ID NO: 1. In these aspects, the IgV ECD is of murine origin.

In aspects, the IgV ECD comprises or consists of a polypeptide having at least 80% sequence identity to SEQ ID NO:2:

MARAMAAAWP LLLVALLVLS WPPPGTGDVV VQAPTQVPGF
LGDSVTLPCY LQVPNMEVTH VSQLTWARHG ESGSMAVFHQ
TQGPSYSESK RLEFVAARLG AELRNASLRM FGLRVEDEGN
YTCLFVTFPQ GSRSVDIWLR VLAKPQNTAE VQKVQLTGEP
VPMARCVSTG GRPPAQITWH SDLGGMPNTS QVPGFLSGTV
TVTSLWILVP SSQVDGKNVT CKVEHESFEK PQLLTVNLTV
YYPPEVSISG YDNNWYLGQN EATLTCDARS NPEPTGYNWS
TTMGPLPPFA VAQGAQLLIR PVDKPINTTL ICNVTNALGA
RQAELTVQVK EGPPSEHSGI SRNAIIFLVL GILVFLILLG
IGIYFYWSKC SREVLWHCHL CPSSTEHASA SANGHVSYSA
VSRENSSSQD PQTEGTR

or a fragment thereof. In other aspects, the IgV ECD comprises or consists of a polypeptide having at least 85, 90, 95, 96, 97, 98, or 99% identity to SEQ ID NO:2, or a fragment thereof. In aspects, the truncated IgV ECD comprises or consists of residues 28-143 of SEQ ID NO: 2. In these aspects, the IgV ECD is of human origin.

The terms “Fc”, “Fc region” or “Fc domain”, may be used interchangeably, and are meant to refer to the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Thus, the Fc domain can be from IgA, IgD, IgG, IgE or IgM, but, in typical aspects, the Fc domain is from IgG, including IgG subclasses IgG1, IgG2, IgG3 and IgG4. In aspects, the Fc domain is an IgG1 Fc domain, in other aspects the Fc domain is an IgG4 Fc domain, and in further aspects, the IgG4 Fc domain includes the hinge region. Similar to the description for IgV ECD, the Fc domain can be of any mammalian source, such as, humans, other higher primates, domestic and farm animals, and zoo, sports, or pet animals, such as mice, dogs, cats, cattle, horses, sheep, pigs, goats, or rabbits. In aspects, the IgG1 Fc domain is a human IgG1 Fc domain, and in other aspects, the IgG1 Fc domain is a mouse IgG1 Fc domain. In some aspects, the IgG4 Fc domain is a human IgG4 Fc domain, and in other aspects, the IgG4 Fc domain is a mouse IgG4 Fc domain.

In aspects, the IgG1 Fc domain comprises or consists of a polypeptide having at least 80% sequence identity to SEQ ID NO:3:

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPGK

or a fragment thereof. In other aspects, the IgG1 Fc domain comprises or consists of a polypeptide having at least 85, 90, 95, 96, 97, 98, or 99% identity to SEQ ID NO:3, or a fragment thereof. In specific aspects, the IgG1 Fc domain comprises or consists of residues 100 to 330 of SEQ ID NO:3. In these aspects, the IgG1 Fc domain is of human origin.

In other aspects, the IgG4 Fc domain comprises or consists of a polypeptide having at least 80% sequence identity to SEQ ID NO:4:

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRVES KYGPPCPSCP APEFLGGPSV
FLFPPKPKDT LMISRTPEVT CVVVDVSQED PEVQFNWYVD
GVEVHNAKTK PREEQFNSTY RVVSVLTVLH QDWLNGKEYK
CKVSNKGLPS SIEKTISKAK GQPREPQVYT LPPSQEEMTK
NQVSLTCLVK GFYPSDIAVE WESNGQPENN YKTTPPVLDS
DGSFFLYSRL TVDKSRWQEG NVFSCSVMHE ALHNHYTQKS
LSLSLGK

or a fragment thereof. In other aspects, the IgG4 Fc domain comprises or consists of a polypeptide having at least 85, 90, 95, 96, 97, 98, or 99% identity to SEQ ID NO:4, or a fragment thereof. In specific aspects, the IgG4 Fc domain comprises or consists of residues 99 to 327 of SEQ ID NO:4. In these aspects, the IgG4 Fc domain is of human origin.

It will be understood that the IgV ECD may be at or near the N-terminus or the C-terminus of the construct and the IgG1 Fc domain would be at or near the corresponding C-terminus or N-terminus. Typically, however, the IgV ECD is at or near the N-terminus of the construct and the Fc domain is at or near the C-terminus of the construct.

In aspects, the construct further comprises a linker. For example, the linker may be included between the PVR and the Fc domain. Alternatively, the linker may be included between the IgV ECD and the Fc domain. In typical aspects, the linker comprises or consists of the sequence IEGRMDP (SEQ ID NO:5)

In aspects, the construct described herein further comprises a secretion signal. The secretion signal sequence component can direct translocation of expressed polypeptides across a membrane. In general, the signal sequence may be a component of the vector, or it may be a part of the target polypeptide DNA that is inserted into the vector. The signal sequence selected is typically one that is recognized and processed (i.e. cleaved by a signal peptidase) by the host cell. In aspects, the secretion signal comprises or consists of the sequence

(SEQ ID NO: 6)
METDTLLLWVLLLWVPGSTG

In specific aspects, the construct described herein comprises or consists of the following sequence (SEQ ID NO: 7):

METDTLLLWVLLLWVPGSTGDIRVLVPYNSTGVLGGSTTLHCSLTSNENV
TITQITWMKKDSGGSHALVAVFHPKKGPNIKEPERVKFLAAQQDLRNASL
AISNLSVEDEGIYECQIATFPRGSRSTNAWLKVQARPKNIEGRMDPTHTC
PPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCS
VMHEALHNHYTQKSLSLSPGK

In the above example, the underlined text is secretion signal, the bold text is PVR IgV ECD, the italicized font is the linker, and the remaining text is the IgG1 Fc domain. In this example, the PVR IgV ECD is of murine origin, and the IgG1 Fc domain is of human origin.

In another specific aspect, the construct described herein comprises or consists of the following sequence (SEQ ID NO:8):

METDTLLLWVLLLWVPGSTGPGTGDVVVQAPTQVPGFLGDSVTLPCYLQV
PNMEVTHVSQLTWARHGESGSMAVFHQTQGPSYSESKRLEFVAARLGAEL
RNASLRMFGLRVEDEGNYTCLFVTFPQGSRSVDIWLESKYGPPCPSCPAP
EFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEAL
HNHYTQKSLSLSLGK

In the above example, the underlined text is secretion signal, the bold text is PVR IgV ECD, and the remaining text is the IgG4 Fc domain, including the hinge region. In this example, the PVR IgV ECD and the IgG4 Fc domain are of human origin.

Sequences that are substantially identical to the above sequences are also contemplated, such as those that are at least about 80, 81, 82, 83, 84, 85, 86, 87, 88, 89, 90, 91, 92, 93, 94, 95, 96, 97, 98, or 99% identical. Fragments of the sequences or the substantially identical variant sequences are also contemplated herein.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The constructs described herein may also comprise additional sequences to aid in their expression, detection or purification. Any such sequences or tags known to those of skill in the art may be used. For example, and without wishing to be limiting, the constructs may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK (SEQ ID NO:13), Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a His₅ or His₆), or a combination thereof. In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

In other aspects, the constructs may be linked to a cargo molecule; the constructs may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer; an enzyme; an anti-cancer antibody for immunotherapy; or any other suitable therapeutic molecule known in the art. In the alternative, a diagnostic agent may include, but is by no means limited to a radioisotope, a paramagnetic label such as gadolinium or iron oxide, a fluorophore, a Near Infra-Red (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, the construct may be linked to a fluorescent agent such as FITC or may genetically be fused to the Enhanced Green Fluorescent Protein (EGFP).

The construct described herein is typically soluble, and, in aspects, binds to a cognate receptor. In other words, the construct described herein can specifically bind to a target (e.g. the cognate receptor) with a particular affinity and/or avidity. Affinity, represented by the equilibrium constant for the dissociation of an antigen with an antibody (K_{D}), measures the binding strength between an antigenic determinant (epitope) and an antibody binding site. Avidity is the measure of the strength of binding between an antibody with its antigen. The lesser the value of the K_{D}, the stronger the binding strength between an antigenic determinant and the antibody binding site. In aspects, the construct described herein binds to the cognate receptor with similar nanomolar affinities (e.g. K_{D}) when compared to the construct comprising the full-length PVR ECD.

The above-noted target (e.g. the cognate receptor) may be any suitable receptor to which the construct described herein binds to. In typical aspects, the cognate receptor or immunomodulator that the construct binds is T cell immunoreceptor with Ig and ITIM domains (TIGIT), CD96 ((Cluster of Differentiation 96; or TACTILE (T cell activation, increased late expression)) and/or CD226 (also known as DNAX Accessory Molecule-1, DNAM-1, PTA-1, TLiSA1).

When compared to constructs comprising full-length PVR ECD, the construct comprising the truncated IgV ECD described herein can bind to TIGIT, CD96 and/or CD226 with similar affinity as compared thereto. In aspects, the construct described herein can bind to the cognate receptor at low nanomolar affinities (e.g. K_{D}), such as, about 0.1 nM to about 10 nM. For example, the construct described herein can bind to the cognate receptor at about 0.1 nM, about 0.2 nM, about 0.3 nM, about 0.4 nM, about 0.5 nM, about 0.6 nM, about 0.7 nM, about 0.8 nM, about 0.9 nM, about 1 nM, about, 2 nM, about 3 nM, about 4 nM, about 5 nM, about 6 nM, about 7 nM, about 8 nM, or about 9 nM, to about about 0.2 nM, about 0.3 nM, about 0.4 nM, about 0.5 nM, about 0.6 nM, about 0.7 nM, about 0.8 nM, about 0.9 nM, about 1 nM, about, 2 nM, about 3 nM, about 4 nM, about 5 nM, about 6 nM, about 7 nM, about 8 nM, about 9 nM, or about 10 nM.

In aspects, the construct binds TIGIT with a nanomolar affinity of about 1 nM to about 3 nM, such as, about 1 nM, about 1.25 nM, about 1.5 nM, about 1.75 nM, about 2 nM, about 2.25 nM, about 2.5 nM or about 2.75 nM, to about about 1.25 nM, about 1.5 nM, about 1.75 nM, about 2 nM, about 2.25 nM, about 2.5 nM about 2.75 nM or about 3 nM. In typical aspects, nanomolar affinity for TIGIT is about 2.2 nM. In other aspects, the construct binds CD96 with a nanomolar affinity of about 1 nM to about 2 nM, such as about 1 nM, about 1.25 nM, about 1.5 nM, or about 1.75 nM, to about 1.25 nM, about 1.5 nM, about 1.75 nM or about 2 nM. In typical aspects, the nanomolar affinity for CD96 is about 1.7 nM. In other aspects, the construct binds CD226 with a nanomolar affinity of about 0.1 nM to about 1 nM, such as about 0.1 nM, about 0.2 nM, about 0.3 nM, about 0.4 nM, about 0.5 nM, about 0.6 nM, about 0.7 nM, about 0.8 nM, or about 0.9 nM, to about 0.2 nM, about 0.3 nM, about 0.4 nM, about 0.5 nM, about 0.6 nM, about 0.7 nM, about 0.8 nM, about 0.9 nM or about 1 nM. In typical aspects, the nanomolar affinity for CD226 is about 0.7 nM.

The construct described herein can suppress immune responses. The suppression of the immune response may be in vivo, when or if the construct described herein is administered to a subject, or the suppression of the immune response may be in vitro, when or if the construct described herein is applied, for example, to a culture of cells. The immune response may be a cytokine response, such that the construct described herein is capable of suppressing cytokine secretion from an immune cell. The cytokine may be any secretable cytokine, such as for example, IFNα, IFNβ, IFNγ, TNF-α, TNF-β, TGF-β, IL-1α, IL-1β, IL-2, IL-3, IL-4, IL-6, IL-8, IL-10, IL-12β, IL-13, IL-17, IL-23, and, GM-CSF, G-CSF, M-CSF, and the like, but in typical aspects, the cytokine of the cytokine response is IL-2, IFNγ, and/or TNFα.

In other aspects, the immune response comprises proliferation of immune cells, such that the construct described herein is capable of suppressing the proliferation of immune cells. In aspects, the construct suppresses proliferation of immune cells by binding to the cognate receptor, such as TIGIT.

The immune cells can be any immune cell, such as, T cells, B cells, monocytes, macrophages, dendritic cells, granulocytes (e.g. eosinophils, basophils and neutrophils) and/or Natural Killer cells (NK cells). In typical aspects, the immune cells comprise T cells (e.g. CD4+ and/or CD8+) and/NK cells, such that, in aspects, the construct described herein reduces the proliferation of T cell and/or NK cells, and in other aspects, the construct described herein reduces cytokine secretion from T cell and/or NK cells. In most typical aspects, the immune cell is the T cell, and the T cell is a CD4⁺ expressing T cell or a CD8⁺ expressing T cell.

The construct described herein can, in aspect, reduce symptoms associated with inflammatory diseases or conditions. The inflammatory disease or condition, can be, for example, Crohn's disease, ulcerative colitis, multiple sclerosis, asthma, rheumatoid arthritis, or psoriasis. In typical aspects, the inflammatory disease or condition is Crohn's disease or ulcerative colitis. In aspects, when the inflammatory disease or condition is Crohn's disease or ulcerative colitis, the symptoms that are reduced by the construct described herein include, for example, weight loss and/or disease severity associated with colitis. In other typical aspects, the inflammatory disease or condition is psoriasis, and in these aspects, the symptoms that are reduced by the construct described herein include, for example, erythema, scaling and/or thickness. In aspects, the construct described herein delays the onset of psoriasis.

Also encompassed herein are isolated or purified polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

Also described herein are nucleic acid molecules encoding the constructs described herein, as well as vectors comprising the nucleic acid molecules and host cells comprising the vectors.

Polynucleotides encoding the constructs described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

In a specific aspect, a polynucleotide encoding the construct described herein has the following sequence (SEQ ID NO:9):

ATGGAAACCGACACACTGCTGCTGTGGGTGCTGCTTCTTTGGGTGCCCGG
AAGCACCGGCGATATCAGAGTGCTGGTGCCCTACAATTCCACCGGCGTGC
TCGGCGGATCTACCACACTGCACTGTAGCCTGACCAGCAACGAGAACGTG
ACCATCACACAGATCACCTGGATGAAGAAGGACAGCGGCGGCTCTCATGC
TCTGGTGGCCGTGTTTCACCCTAAGAAGGGCCCCAACATCAAAGAACCCG
AGAGAGTGAAGTTCCTGGCCGCTCAGCAGGACCTGAGAAATGCCTCTCTG
GCCATCAGCAACCTGAGCGTGGAAGATGAGGGCATCTACGAGTGCCAGAT
CGCCACCTTTCCTAGAGGCAGCAGAAGCACCAACGCCTGGCTGAAGGTTC
AGGCCAGACCTAAGAACATCGAGGGCAGAATGGACCCCACACACACCTGT
CCTCCATGTCCTGCTCCAGAGCTGCTCGGAGGCCCTTCCGTGTTTCTGTT
CCCTCCAAAGCCTAAGGACACCCTGATGATCAGCAGAACCCCTGAAGTGA
CCTGCGTGGTGGTGGATGTGTCCCACGAGGATCCCGAAGTGAAGTTTAAT
TGGTACGTGGACGGCGTGGAAGTGCACAACGCCAAGACCAAGCCTAGAGA
GGAACAGTACAACAGCACCTACAGAGTGGTGTCCGTGCTGACCGTGCTGC
ACCAGGATTGGCTGAACGGCAAAGAGTACAAGTGCAAGGTGTCCAACAAG
GCCCTGCCTGCTCCTATCGAGAAAACCATCTCCAAGGCCAAGGGCCAGCC
TAGGGAACCCCAGGTTTACACACTGCCTCCAAGCCGGGAAGAGATGACCA
AGAACCAGGTGTCCCTGACCTGCCTGGTCAAGGGCTTCTACCCTAGCGAC
ATTGCCGTGGAATGGGAGAGCAATGGCCAGCCTGAGAACAACTACAAGAC
CACACCTCCTGTGCTGGACAGCGACGGCTCATTCTTCCTGTACAGCAAGC
TGACAGTGGACAAGAGCAGATGGCAGCAGGGCAACGTGTTCAGCTGCAGC
GTGATGCACGAGGCCCTGCACAACCACTACACCCAGAAGTCCCTGAGCCT
GTCTCCTGGCAAGTGATGATGAA

or a sequence at least 80% identical thereto, or a fragment thereof.

In the above specific aspect, the construct is defined by DNA sequence and has the mouse IgV ECD of PVR (e.g. IgV PVR-Fc or vdPVR-Fc).

In another specific aspect, a polynucleotide encoding the construct described herein has the following sequence (SEQ ID NO:10):

AUGGAAACCGACACACUGCUGCUGUGGGUGCUGCUUCUUUGGGUGCCCGG
AAGCACCGGCGAUAUCAGAGUGCUGGUGCCCUACAAUUCCACCGGCGUGC
UCGGCGGAUCUACCACACUGCACUGUAGCCUGACCAGCAACGAGAACGUG
ACCAUCACACAGAUCACCUGGAUGAAGAAGGACAGCGGCGGCUCUCAUGC
UCUGGUGGCCGUGUUUCACCCUAAGAAGGGCCCCAACAUCAAAGAACCCG
AGAGAGUGAAGUUCCUGGCCGCUCAGCAGGACCUGAGAAAUGCCUCUCUG
GCCAUCAGCAACCUGAGCGUGGAAGAUGAGGGCAUCUACGAGUGCCAGAU
CGCCACCUUUCCUAGAGGCAGCAGAAGCACCAACGCCUGGCUGAAGGUUC
AGGCCAGACCUAAGAACAUCGAGGGCAGAAUGGACCCCACACACACCUGU
CCUCCAUGUCCUGCUCCAGAGCUGCUCGGAGGCCCUUCCGUGUUUCUGUU
CCCUCCAAAGCCUAAGGACACCCUGAUGAUCAGCAGAACCCCUGAAGUGA
CCUGCGUGGUGGUGGAUGUGUCCCACGAGGAUCCCGAAGUGAAGUUUAAU
UGGUACGUGGACGGCGUGGAAGUGCACAACGCCAAGACCAAGCCUAGAGA
GGAACAGUACAACAGCACCUACAGAGUGGUGUCCGUGCUGACCGUGCUGC
ACCAGGAUUGGCUGAACGGCAAAGAGUACAAGUGCAAGGUGUCCAACAAG
GCCCUGCCUGCUCCUAUCGAGAAAACCAUCUCCAAGGCCAAGGGCCAGCC
UAGGGAACCCCAGGUUUACACACUGCCUCCAAGCCGGGAAGAGAUGACCA
AGAACCAGGUGUCCCUGACCUGCCUGGUCAAGGGCUUCUACCCUAGCGAC
AUUGCCGUGGAAUGGGAGAGCAAUGGCCAGCCUGAGAACAACUACAAGAC
CACACCUCCUGUGCUGGACAGCGACGGCUCAUUCUUCCUGUACAGCAAGC
UGACAGUGGACAAGAGCAGAUGGCAGCAGGGCAACGUGUUCAGCUGCAGC
GUGAUGCACGAGGCCCUGCACAACCACUACACCCAGAAGUCCCUGAGCCU
GUCUCCUGGCAAGUGAUGAUGAA

or a sequence at least 80% identical thereto, or a fragment thereof.

In the above specific aspect, the construct is defined by mRNA sequence and has the mouse IgV ECD of PVR (e.g. IgV PVR-Fc or vdPVR-Fc).

In another specific aspect, a polynucleotide encoding the construct described herein has the following sequence (SEQ ID NO:11):

ATGGAAACCGACACACTGCTGCTGTGGGTGCTGCTTCTTTGGGTGCCCGG
ATCTACAGGACCTGGAACAGGCGACGTTGTGGTGCAGGCTCCTACTCAGG
TGCCAGGCTTTCTGGGCGATAGCGTGACCCTGCCTTGCTACCTGCAAGTG
CCCAACATGGAAGTGACCCACGTGTCCCAGCTGACCTGGGCTAGACATGG
CGAGTCTGGATCCATGGCCGTGTTCCACCAGACACAGGGCCCTAGCTACA
GCGAGAGCAAGCGGCTGGAATTTGTGGCCGCTAGACTGGGAGCCGAGCTG
AGAAATGCCAGCCTGAGAATGTTCGGCCTGAGAGTGGAAGATGAGGGCAA
CTACACCTGTCTGTTCGTGACCTTTCCACAGGGCAGCAGAAGCGTGGACA
TCTGGCTGGAATCTAAGTACGGCCCTCCTTGTCCTAGCTGCCCCGCTCCT
GAATTTCTCGGCGGACCTTCCGTGTTCCTGTTTCCTCCAAAGCCTAAGGA
CACCCTGATGATCAGCAGAACCCCTGAAGTGACCTGCGTGGTGGTGGATG
TGTCCCAAGAGGATCCCGAGGTGCAGTTCAATTGGTACGTGGACGGCGTG
GAAGTGCACAACGCCAAGACCAAGCCTAGAGAGGAACAGTTCAACAGCAC
CTACAGAGTGGTGTCCGTGCTGACCGTGCTGCACCAGGATTGGCTGAACG
GCAAAGAGTACAAGTGCAAGGTGTCCAACAAGGGCCTGCCTAGCAGCATC
GAGAAAACCATCAGCAAGGCCAAGGGCCAGCCAAGAGAACCCCAGGTGTA
CACACTGCCTCCAAGCCAAGAGGAAATGACCAAGAACCAGGTGTCCCTGA
CCTGCCTGGTCAAGGGCTTCTACCCTTCCGATATCGCCGTGGAATGGGAG
AGCAATGGACAGCCCGAGAACAACTACAAGACCACACCTCCTGTGCTGGA
CAGCGACGGCTCATTCTTCCTGTACAGCAGACTGACCGTGGACAAGAGCA
GATGGCAAGAGGGCAACGTGTTCAGCTGCAGCGTGATGCACGAGGCCCTG
CACAACCACTACACCCAGAAGTCTCTGAGCCTGAGCCTGGGCAAGTGATG
ATGA

or a sequence at least 80% identical thereto, or a fragment thereof.

In the above specific aspect, the construct is defined by DNA sequence and has the human IgV ECD of PVR (e.g. IgV PVR-Fc or vdPVR-Fc).

In further specific aspect, a polynucleotide encoding the construct described herein has the following sequence (SEQ ID NO:12):

AUGGAAACCGACACACUGCUGCUGUGGGUGCUGCUUCUUUGGGUGCCCGG
AUCUACAGGACCUGGAACAGGCGACGUUGUGGUGCAGGCUCCUACUCAGG
UGCCAGGCUUUCUGGGCGAUAGCGUGACCCUGCCUUGCUACCUGCAAGUG
CCCAACAUGGAAGUGACCCACGUGUCCCAGCUGACCUGGGCUAGACAUGG
CGAGUCUGGAUCCAUGGCCGUGUUCCACCAGACACAGGGCCCUAGCUACA
GCGAGAGCAAGCGGCUGGAAUUUGUGGCCGCUAGACUGGGAGCCGAGCUG
AGAAAUGCCAGCCUGAGAAUGUUCGGCCUGAGAGUGGAAGAUGAGGGCAA
CUACACCUGUCUGUUCGUGACCUUUCCACAGGGCAGCAGAAGCGUGGACA
UCUGGCUGGAAUCUAAGUACGGCCCUCCUUGUCCUAGCUGCCCCGCUCCU
GAAUUUCUCGGCGGACCUUCCGUGUUCCUGUUUCCUCCAAAGCCUAAGGA
CACCCUGAUGAUCAGCAGAACCCCUGAAGUGACCUGCGUGGUGGUGGAUG
UGUCCCAAGAGGAUCCCGAGGUGCAGUUCAAUUGGUACGUGGACGGCGUG
GAAGUGCACAACGCCAAGACCAAGCCUAGAGAGGAACAGUUCAACAGCAC
CUACAGAGUGGUGUCCGUGCUGACCGUGCUGCACCAGGAUUGGCUGAACG
GCAAAGAGUACAAGUGCAAGGUGUCCAACAAGGGCCUGCCUAGCAGCAUC
GAGAAAACCAUCAGCAAGGCCAAGGGCCAGCCAAGAGAACCCCAGGUGUA
CACACUGCCUCCAAGCCAAGAGGAAAUGACCAAGAACCAGGUGUCCCUGA
CCUGCCUGGUCAAGGGCUUCUACCCUUCCGAUAUCGCCGUGGAAUGGGAG
AGCAAUGGACAGCCCGAGAACAACUACAAGACCACACCUCCUGUGCUGGA
CAGCGACGGCUCAUUCUUCCUGUACAGCAGACUGACCGUGGACAAGAGCA
GAUGGCAAGAGGGCAACGUGUUCAGCUGCAGCGUGAUGCACGAGGCCCUG
CACAACCACUACACCCAGAAGUCUCUGAGCCUGAGCCUGGGCAAGUGAUG
AUGA

or a sequence at least 80% identical thereto, or a fragment thereof.

In the above specific aspect, the construct is defined by mRNA sequence and has the human IgV ECD of PVR (e.g. IgV PVR-Fc or vdPVR-Fc).

Additionally, expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

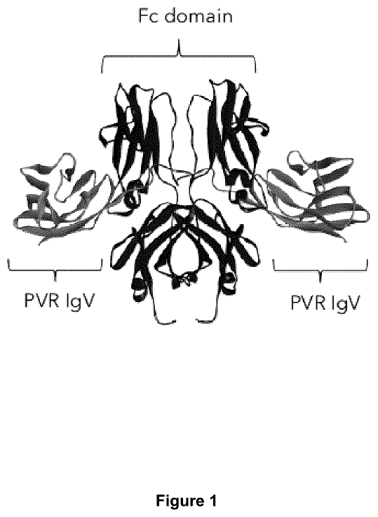

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as MI3 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA. In aspects, the construct described herein is expressed as a secreted soluble protein in Expi293 mammalian cells, and later purified using techniques known in the art (e.g. Protein A affinity chromatography). In aspects, the construct described herein produces as a stable dimer in Expi293 mammalian cells (FIG. 1).

Additional eukaryotic expression vectors are known in the art (e.g., P J. Southern & P. Berg, J. Mol. Appl. Genet, 1:327-341 (1982); Subramani et al, Mol. Cell. Biol, 1:854-864 (1981); Kaufinann & Sharp, “Amplification and Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The constructs described herein can be expressed in cell lines other than in hybridomas. Nucleic acids, which comprise a sequence encoding a polypeptide, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce proteins by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6):654-664, Nielsen et al, Prot. Eng., 10:1-6 (1997); von Heinje et al., Nucl. Acids Res., 14:4683-4690 (1986), all of which are incorporated by reference herein) at the 5′ end of the protein-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium. In exemplary aspects, the recombinant protein described herein can be expressed by commensal bacteria, such as Lactococcus lactis, such that these bacteria express and deliver the expressed protein (e.g. therapeutic protein) directly to the gut to treat inflammatory events related to inflammatory bowel disease (IBD). In these aspects, the commensal strains can be made to secrete proteins while they transit through the gut, for example.

The constructs described herein can be fused to additional amino acid residues. Such amino acid residues can be a peptide tag to facilitate isolation, for example. Other amino acid residues for homing of the antibodies to specific organs or tissues are also contemplated.

Also described herein are methods for suppressing an immune response. The suppression of the immune response may be in vitro or in vivo as described above. In other aspects, methods of treating an inflammatory disease or condition are also provided. The methods comprise administering the construct described herein to a subject in need thereof.

Also described herein are uses of the construct described herein for supressing an immune response in vivo or in vitro. In addition, uses of the construct described herein for treating and/or preventing an inflammatory disease or condition and use of the construct described herein for reducing symptoms associated with an inflammatory disease or condition are also provided. In these aspects, the construct described herein is for administration to the subject in need thereof.

The immune response can be the cytokine response or immune cell proliferation described herein, and the immune cells and the cytokines are as described above. In typical aspects, the immune cell is the T cell (e.g. CD4⁺ and/or CD8⁺) described herein, and the cytokine is IL-2 and/or INFγ. In typical aspects, the reduction of proliferation of T cells is accomplished by binding of the construct described herein to TIGIT as described herein. In respect of the inflammatory disease or condition, in typical aspects, the inflammatory disease or condition is Crohn's disease or ulcerative colitis, and typically, the inflammatory disease or condition is ulcerative colitis. In other typical aspects, the inflammatory disease or condition is psoriasis. When the inflammatory disease is Crohn's disease or ulcerative colitis, the symptom is typically weight loss and/or disease severity associated with colitis, and when the inflammatory disease or condition is psoriasis, the symptom is typically erythema, scaling and/or thickness.

The subject in the methods and uses described herein includes, but is not limited to, humans, laboratory animals, domestic pets and farm animals. In typical aspects, the subject is a human. In order to treat the subject, a therapeutically effective dose of the construct described herein may be administered. “Therapeutically effective dose” refers to a dose that produces the effects for which it is administered. The exact dose will depend on the purpose of the treatment, and will be ascertainable by one skilled in the art using known techniques. Any suitable method or route can be used to administer the constructs described herein. Routes of administration include, for example, oral, intravenous, intraperitoneal, subcutaneous, or intramuscular administration.

It is understood that the constructs described herein, where used in a mammal for the purpose of prophylaxis or treatment, can be administered in the form of a composition, such as, for example, a sterile aqueous solution, additionally comprising a pharmaceutically acceptable carrier. Suitable pharmaceutically acceptable carriers include, for example, one or more of water, saline, phosphate buffered saline, dextrose, glycerol, ethanol and the like (see, generally, Remington's Pharmaceutical Sciences 16^{th} Edition, A. Osal., Ed., 1980), as well as combinations thereof. Acceptable carriers, excipients, or stabilizers are nontoxic to recipients at the dosages and concentrations employed and may include buffers. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal. Thus, in aspects, the constructs described, including the polynucleotide sequences described herein, may be used as therapeutics, such as LNP-packaged RNA therapeutics (e.g. COVID vaccines).

The dosing amounts and frequencies of administration are, in aspects, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

Also included herein are kits, comprising a therapeutically or prophylactically effective amount of the constructs described herein. The kits can further contain any suitable components, including buffers, for example. Kits may include instructions.

Sequence Listing:

Mouse IgV ECD SEQ ID NO: 1:
MAQLARATRS PLSWLLLLFC YALRKAGGDI RVLVPYNSTG VLGGSTTLHC SLTSNENVTI
TQITWMKKDS GGSHALVAVF HPKKGPNIKE PERVKFLAAQ QDLRNASLAI SNLSVEDEGI
YECQIATFPR GSRSTNAWLK VQARPKNTAE ALEPSPTLIL QDVAKCISAN GHPPGRISWP
SNVNGSHREM KEPGSQPGTT TVTSYLSMVP SRQADGKNIT CTVEHESLQE LDQLLVTLSQ
PYPPENVSIS GYDGNWYVGL TNLTLTCEAH SKPAPDMAGY NWSTNTGDFP NSVKRQGNML
LISTVEDGLN NTVIVCEVTN ALGSGQGQVH IIVKEKPENM QQNTRLHLGY IFLIVFVLAV
VIIIAALYTI RRCRHGRALQ SNPSERENVQ YSSVNGDCRL NMEPNSTR
Human IgV ECD SEQ ID NO: 2:
MARAMAAAWP LLLVALLVLS WPPPGTGDVV VQAPTQVPGF LGDSVTLPCY LQVPNMEVTH
VSQLTWARHG ESGSMAVFHQ TQGPSYSESK RLEFVAARLG AELRNASLRM FGLRVEDEGN
YTCLFVTFPQ GSRSVDIWLR VLAKPQNTAE VQKVQLTGEP VPMARCVSTG GRPPAQITWH
SDLGGMPNTS QVPGFLSGTV TVTSLWILVP SSQVDGKNVT CKVEHESFEK PQLLTVNLTV
YYPPEVSISG YDNNWYLGQN EATLTCDARS NPEPTGYNWS TTMGPLPPFA VAQGAQLLIR
PVDKPINTTL ICNVTNALGA RQAELTVQVK EGPPSEHSGI SRNAIIFLVL GILVFLILLG
IGIYFYWSKC SREVLWHCHL CPSSTEHASA SANGHVSYSA VSRENSSSQD PQTEGTR
Human IgG1 Fc domain SEQ ID NO: 3.
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKFNW YVDGVEVHNA KTKPREEQYN
STYRVVSVLT VLHQDWLNGK EYKCKVSNKA LPAPIEKTIS KAKGQPREPQ VYTLPPSRDE
LTKNQVSLTC LVKGFYPSDI AVEWESNGQP ENNYKTTPPV LDSDGSFFLY SKLTVDKSRW
QQGNVFSCSV MHEALHNHYT QKSLSLSPGK
Human IgG4 Fc domain SEQ ID NO: 4.
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPSCP APEFLGGPSV
FLFPPKPKDT LMISRTPEVT CVVVDVSQED PEVQFNWYVD GVEVHNAKTK PREEQFNSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGK
Linker SEQ ID NO: 5:
IEGRMDP
Secretion signal SEQ ID NO: 6:
METDTLLLWVLLLWVPGSTG
Mouse IgV ECD in vdPVR-Fc SEQ ID NO: 7:
METDTLLLWVLLLWVPGSTGDIRVLVPYNSTGVLGGSTTLHCSLTSNENVTITQITWMKKDSGG
SHALVAVFHPKKGPNIKEPERVKFLAAQQDLRNASLAISNLSVEDEGIYECQIATFPRGSRSTNA
WLKVQARPKNIEGRMDPTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
Human IgV ECD in vdPVR-Fc SEQ ID NO: 8:
METDTLLLWVLLLWVPGSTGPGTGDVVVQAPTQVPGFLGDSVTLPCYLQVPNMEVTHVSQLT
WARHGESGSMAVFHQTQGPSYSESKRLEFVAARLGAELRNASLRMFGLRVEDEGNYTCLFVT
FPQGSRSVDIWLESKYGPPCPSCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGK
Mouse IgV ECD in vdPVR-Fc (DNA) SEQ ID NO: 9:
ATGGAAACCGACACACTGCTGCTGTGGGTGCTGCTTCTTTGGGTGCCCGGAAGCACCGGC
GATATCAGAGTGCTGGTGCCCTACAATTCCACCGGCGTGCTCGGCGGATCTACCACACTG
CACTGTAGCCTGACCAGCAACGAGAACGTGACCATCACACAGATCACCTGGATGAAGAAG
GACAGCGGCGGCTCTCATGCTCTGGTGGCCGTGTTTCACCCTAAGAAGGGCCCCAACATC
AAAGAACCCGAGAGAGTGAAGTTCCTGGCCGCTCAGCAGGACCTGAGAAATGCCTCTCTG
GCCATCAGCAACCTGAGCGTGGAAGATGAGGGCATCTACGAGTGCCAGATCGCCACCTTT
CCTAGAGGCAGCAGAAGCACCAACGCCTGGCTGAAGGTTCAGGCCAGACCTAAGAACATC
GAGGGCAGAATGGACCCCACACACACCTGTCCTCCATGTCCTGCTCCAGAGCTGCTCGGA
GGCCCTTCCGTGTTTCTGTTCCCTCCAAAGCCTAAGGACACCCTGATGATCAGCAGAACCC
CTGAAGTGACCTGCGTGGTGGTGGATGTGTCCCACGAGGATCCCGAAGTGAAGTTTAATT
GGTACGTGGACGGCGTGGAAGTGCACAACGCCAAGACCAAGCCTAGAGAGGAACAGTAC
AACAGCACCTACAGAGTGGTGTCCGTGCTGACCGTGCTGCACCAGGATTGGCTGAACGGC
AAAGAGTACAAGTGCAAGGTGTCCAACAAGGCCCTGCCTGCTCCTATCGAGAAAACCATCT
CCAAGGCCAAGGGCCAGCCTAGGGAACCCCAGGTTTACACACTGCCTCCAAGCCGGGAA
GAGATGACCAAGAACCAGGTGTCCCTGACCTGCCTGGTCAAGGGCTTCTACCCTAGCGAC
ATTGCCGTGGAATGGGAGAGCAATGGCCAGCCTGAGAACAACTACAAGACCACACCTCCT
GTGCTGGACAGCGACGGCTCATTCTTCCTGTACAGCAAGCTGACAGTGGACAAGAGCAGA
TGGCAGCAGGGCAACGTGTTCAGCTGCAGCGTGATGCACGAGGCCCTGCACAACCACTA
CACCCAGAAGTCCCTGAGCCTGTCTCCTGGCAAGTGATGATGAA
Mouse IgV ECD in vdPVR-Fc (mRNA) SEQ ID NO: 10:
AUGGAAACCGACACACUGCUGCUGUGGGUGCUGCUUCUUUGGGUGCCCGGAAGCACCG
GCGAUAUCAGAGUGCUGGUGCCCUACAAUUCCACCGGCGUGCUCGGCGGAUCUACCAC
ACUGCACUGUAGCCUGACCAGCAACGAGAACGUGACCAUCACACAGAUCACCUGGAUGA
AGAAGGACAGCGGCGGCUCUCAUGCUCUGGUGGCCGUGUUUCACCCUAAGAAGGGCCC
CAACAUCAAAGAACCCGAGAGAGUGAAGUUCCUGGCCGCUCAGCAGGACCUGAGAAAUG
CCUCUCUGGCCAUCAGCAACCUGAGCGUGGAAGAUGAGGGCAUCUACGAGUGCCAGAU
CGCCACCUUUCCUAGAGGCAGCAGAAGCACCAACGCCUGGCUGAAGGUUCAGGCCAGA
CCUAAGAACAUCGAGGGCAGAAUGGACCCCACACACACCUGUCCUCCAUGUCCUGCUCC
AGAGCUGCUCGGAGGCCCUUCCGUGUUUCUGUUCCCUCCAAAGCCUAAGGACACCCUG
AUGAUCAGCAGAACCCCUGAAGUGACCUGCGUGGUGGUGGAUGUGUCCCACGAGGAUC
CCGAAGUGAAGUUUAAUUGGUACGUGGACGGCGUGGAAGUGCACAACGCCAAGACCAA
GCCUAGAGAGGAACAGUACAACAGCACCUACAGAGUGGUGUCCGUGCUGACCGUGCUG
CACCAGGAUUGGCUGAACGGCAAAGAGUACAAGUGCAAGGUGUCCAACAAGGCCCUGC
CUGCUCCUAUCGAGAAAACCAUCUCCAAGGCCAAGGGCCAGCCUAGGGAACCCCAGGUU
UACACACUGCCUCCAAGCCGGGAAGAGAUGACCAAGAACCAGGUGUCCCUGACCUGCCU
GGUCAAGGGCUUCUACCCUAGCGACAUUGCCGUGGAAUGGGAGAGCAAUGGCCAGCCU
GAGAACAACUACAAGACCACACCUCCUGUGCUGGACAGCGACGGCUCAUUCUUCCUGUA
CAGCAAGCUGACAGUGGACAAGAGCAGAUGGCAGCAGGGCAACGUGUUCAGCUGCAGC
GUGAUGCACGAGGCCCUGCACAACCACUACACCCAGAAGUCCCUGAGCCUGUCUCCUG
GCAAGUGAUGAUGAA
Human IgV ECD in vdPVR-Fc (DNA) SEQ ID NO: 11:
ATGGAAACCGACACACTGCTGCTGTGGGTGCTGCTTCTTTGGGTGCCCGGATCTACAGGA
CCTGGAACAGGCGACGTTGTGGTGCAGGCTCCTACTCAGGTGCCAGGCTTTCTGGGCGAT
AGCGTGACCCTGCCTTGCTACCTGCAAGTGCCCAACATGGAAGTGACCCACGTGTCCCAG
CTGACCTGGGCTAGACATGGCGAGTCTGGATCCATGGCCGTGTTCCACCAGACACAGGGC
CCTAGCTACAGCGAGAGCAAGCGGCTGGAATTTGTGGCCGCTAGACTGGGAGCCGAGCT
GAGAAATGCCAGCCTGAGAATGTTCGGCCTGAGAGTGGAAGATGAGGGCAACTACACCTG
TCTGTTCGTGACCTTTCCACAGGGCAGCAGAAGCGTGGACATCTGGCTGGAATCTAAGTA
CGGCCCTCCTTGTCCTAGCTGCCCCGCTCCTGAATTTCTCGGCGGACCTTCCGTGTTCCT
GTTTCCTCCAAAGCCTAAGGACACCCTGATGATCAGCAGAACCCCTGAAGTGACCTGCGT
GGTGGTGGATGTGTCCCAAGAGGATCCCGAGGTGCAGTTCAATTGGTACGTGGACGGCGT
GGAAGTGCACAACGCCAAGACCAAGCCTAGAGAGGAACAGTTCAACAGCACCTACAGAGT
GGTGTCCGTGCTGACCGTGCTGCACCAGGATTGGCTGAACGGCAAAGAGTACAAGTGCAA
GGTGTCCAACAAGGGCCTGCCTAGCAGCATCGAGAAAACCATCAGCAAGGCCAAGGGCC
AGCCAAGAGAACCCCAGGTGTACACACTGCCTCCAAGCCAAGAGGAAATGACCAAGAACC
AGGTGTCCCTGACCTGCCTGGTCAAGGGCTTCTACCCTTCCGATATCGCCGTGGAATGGG
AGAGCAATGGACAGCCCGAGAACAACTACAAGACCACACCTCCTGTGCTGGACAGCGACG
GCTCATTCTTCCTGTACAGCAGACTGACCGTGGACAAGAGCAGATGGCAAGAGGGCAACG
TGTTCAGCTGCAGCGTGATGCACGAGGCCCTGCACAACCACTACACCCAGAAGTCTCTGA
GCCTGAGCCTGGGCAAGTGATGATGA
Human IgV ECD in vdPVR-Fc (mRNA) SEQ ID NO: 12:
AUGGAAACCGACACACUGCUGCUGUGGGUGCUGCUUCUUUGGGUGCCCGGAUCUACAG
GACCUGGAACAGGCGACGUUGUGGUGCAGGCUCCUACUCAGGUGCCAGGCUUUCUGG
GCGAUAGCGUGACCCUGCCUUGCUACCUGCAAGUGCCCAACAUGGAAGUGACCCACGU
GUCCCAGCUGACCUGGGCUAGACAUGGCGAGUCUGGAUCCAUGGCCGUGUUCCACCAG
ACACAGGGCCCUAGCUACAGCGAGAGCAAGCGGCUGGAAUUUGUGGCCGCUAGACUGG
GAGCCGAGCUGAGAAAUGCCAGCCUGAGAAUGUUCGGCCUGAGAGUGGAAGAUGAGGG
CAACUACACCUGUCUGUUCGUGACCUUUCCACAGGGCAGCAGAAGCGUGGACAUCUGG
CUGGAAUCUAAGUACGGCCCUCCUUGUCCUAGCUGCCCCGCUCCUGAAUUUCUCGGCG
GACCUUCCGUGUUCCUGUUUCCUCCAAAGCCUAAGGACACCCUGAUGAUCAGCAGAACC
CCUGAAGUGACCUGCGUGGUGGUGGAUGUGUCCCAAGAGGAUCCCGAGGUGCAGUUCA
AUUGGUACGUGGACGGCGUGGAAGUGCACAACGCCAAGACCAAGCCUAGAGAGGAACA
GUUCAACAGCACCUACAGAGUGGUGUCCGUGCUGACCGUGCUGCACCAGGAUUGGCUG
AACGGCAAAGAGUACAAGUGCAAGGUGUCCAACAAGGGCCUGCCUAGCAGCAUCGAGAA
AACCAUCAGCAAGGCCAAGGGCCAGCCAAGAGAACCCCAGGUGUACACACUGCCUCCAA
GCCAAGAGGAAAUGACCAAGAACCAGGUGUCCCUGACCUGCCUGGUCAAGGGCUUCUA
CCCUUCCGAUAUCGCCGUGGAAUGGGAGAGCAAUGGACAGCCCGAGAACAACUACAAGA
CCACACCUCCUGUGCUGGACAGCGACGGCUCAUUCUUCCUGUACAGCAGACUGACCGU
GGACAAGAGCAGAUGGCAAGAGGGCAACGUGUUCAGCUGCAGCGUGAUGCACGAGGCC
CUGCACAACCACUACACCCAGAAGUCUCUGAGCCUGAGCCUGGGCAAGUGAUGAUGA

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Without further description, it is believed that one of ordinary skill in the art can, using the preceding description and the following illustrative examples, make and utilize the constructs of the present invention and practice the claimed methods. The following working examples therefore, specifically point out the typical aspects of the present invention and are not to be construed as limiting in any way in the remainder of the disclosure. Although specific terms have been employed herein, such terms are intended in a descriptive sense and not for purposes of limitation.

### EXAMPLES

### Statistic Analyses for the Examples

Statistical analyses were completed using GraphPad Prism 9 statistical software using one-way or two-way ANOVA to analyze mean differences between groups. P-values less than 0.05 were considered statistically significant.

### Example 1. Design and Production of Murine vdPVR-Fc and Mutant vdPVR-Fc Methods

The vdPVR-Fc construct was synthesized by linking the murine PVR IgV ECD (Accession #NP_081790.1; residues 29-147) upstream of the human IgG1 Fc region (Accession #P01857; residues 100-330) and inserted into the pcDNA3.4 TOPO mammalian expression plasmid (GeneArt; Thermo Fisher Scientific). The linker sequence IEGRMDP was inserted between the PVR IgV ECD and the human IgG1 Fc domain. The mutant vdPVR-Fc construct was synthesized similarly except two-point mutations (Q63R and F128R) were inserted in the PVR IgV ECD to abolish binding to TIGIT and CD226. Both plasmids also contained a 5′ Ig-kappa leader sequence (30) for secretion into culture media. Recombinant proteins were produced using Expi293F expression system (GeneArt; Thermo Fisher Scientific). Secreted proteins were purified using HiTrap protein A HP column (17040301; Cytiva) and eluted with about 0.1 M glycine-HCl (pH about 2.7) and neutralized with about 1 M Tris-HCl (pH about 9.0). After purification, protein samples were desalted into PBS using PD10 columns (17085101; Cytiva) and ran through endotoxin removal columns (88274; Thermo Fisher Scientific). Finally, protein samples were verified for purity using SDS-PAGE and Western blot. The blots were probed with a goat anti-human Fc fragment antibody conjugated to horseradish peroxidase (HRP) (A80-304P; Bethyl Laboratories). Protein concentrations were quantified by measuring sample absorbance at 280 nm. The mutant vdPVR-Fc ribbon model was generated using Colabfold (33). The amino acid sequence of the mutant vdPVR-Fc construct was uploaded, and the generated ribbon model was constructed as a homodimer homo-oligomer.

### Results/Discussion

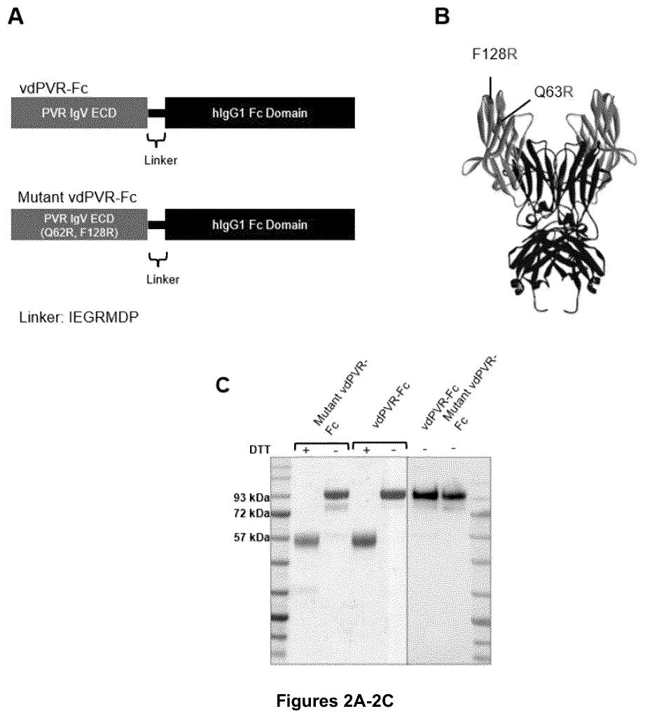

The ribbon structure vdPVR-Fc is shown in FIG. 1 and that of the mutant vdPVR-Fc is shown in FIG. 2B. Recombinant PVR-Fc fusion construct (vdPVR-Fc) was produced by fusing the IgV domain of murine PVR ECD to a human IgG1 Fc domain (FIG. 2A). A similar construct was designed to incorporate two amino acid mutations (Q63R and F128R; FIG. 2A and B), previously shown to abrogate PVR binding to TIGIT and CD226 (13, 14). This construct, termed mutant vdPVR-Fc, served as a negative control. Both constructs were expressed as secreted soluble proteins in Expi293 mammalian cells and purified using Protein A affinity chromatography. The purity and molecular weight of the proteins were confirmed by SDS-PAGE and Western Blot analysis. As shown in FIG. 2C, both proteins migrated at the expected molecular weight for the dimeric form of vdPVR-Fc (about 94 kDa) in the absence of a reducing agent (FIG. 2C).

### References Cited

13. Wang H, et al. Binding mode of the side-by-side two-IgV molecule CD226/DNAM-1 to its ligand CD155/Necl-5. Proc Natl Acad Sci U S A 2019;116(3):988-996.
14. Stengel K F, et al. Structure of TIGIT immunoreceptor bound to poliovirus receptor reveals a cell-cell adhesion and signaling mechanism that requires cis-trans receptor clustering. Proc Natl Acad Sci U S A 2012;109(14):5399-5404.
30. Wang X, et al. Efficient production of CYTL1 protein using mouse IgGk signal peptide in the CHO cell expression system. Acta Biochim Biophys Sin (Shanghai) 2016;48(4):391.
33. Mirdita M, et al. ColabFold: making protein folding accessible to all. Nature Methods 2022 19:6 2022;19(6):679-682.

### Example 2. Assessment of vdPVR-Fc Binding Affinity to TIGIT, CD266 and CD96

The extracellular domain (ECD) of the polio virus receptor (PVR; CD155), expressed on antigen presenting cells and tumor cells, is composed of an N-terminal IgV region followed by two IgC elements. This ECD has been shown to bind to T-cell immunoglobulin and ITIM domain (TIGIT; also known as WUCAM, VSTM3 and VSIG9), a co-inhibitory receptor that is expressed on activated CD4⁺ T-cells, CD8⁺ T-cells and NK-cells, where it suppresses their activity (5, 6). Furthermore, PVR binds to the co-inhibitory receptor CD96 (TACTILE) and the costimulatory receptor CD226 (DNAM1) which are also expressed on T-cells and NK-cells (7, 8). Interestingly, TIGIT inhibits CD226-mediated co-stimulation by blocking CD226 dimerization (9). TIGIT has also been reported to bind to PVR with high affinity relative to CD226 (10). Finally, CD96 has been demonstrated to compete for PVR binding to CD226 to directly inhibit NK-cell functions (7). Thus, the engineered PVR ECD constructs described herein may be able to simultaneously target both TIGIT and CD96 inhibitory pathways, and therefore, could represent a biologic with a potentially broader anti-inflammatory property.

### Methods

The vdPVR, mutant vdPVR were synthesized as described in Example 1.

Surface Plasmon Resonance: Binding kinetics of vdPVR-Fc, mutant vdPVR-Fc and PVR-Fc (786608; Biolegend) proteins to mTIGIT-Fc-his (771808; Biolegend), mCD226-his (50232-M08H; SinoBiological) and mCD96-his (788806; Biolegend) were derived from single cycle kinetic analyses by surface plasmon resonance (SPR) using a Biacore T200 (Cytiva). Briefly, anti-histidine antibodies were immobilized on a CM5 chip (29149604; Cytiva) using the His capture kit (28995056; Cytiva) following manufacturer's protocol. The target proteins (mTIGIT-Fc-His tag, mCD226-His tag and mCD96-His tag were captured on the anti-histidine coated chip by flowing about 1 to about 5 μg/mL of protein at a flow rate of about 30 μL/min. Five concentrations (1:2 serial dilutions) of vdPVR-Fc, mutant vdPVR-Fc and PVR-Fc were flown over the immobilized proteins. All proteins were diluted in HBS-EP running buffer (about 20 mM of HEPES pH about 7.4, about 150 mM NaCl, about 0.005% Tween-20, about 3.4 mM EDTA). The derived sensorgrams were fitted to a 1:1 Langmuir binding model and analyzed using the Biacore T200 evaluation software to calculate the on-rate (ka), off-rate (kd), and the equilibrium constant (KD).

### Results/Discussion

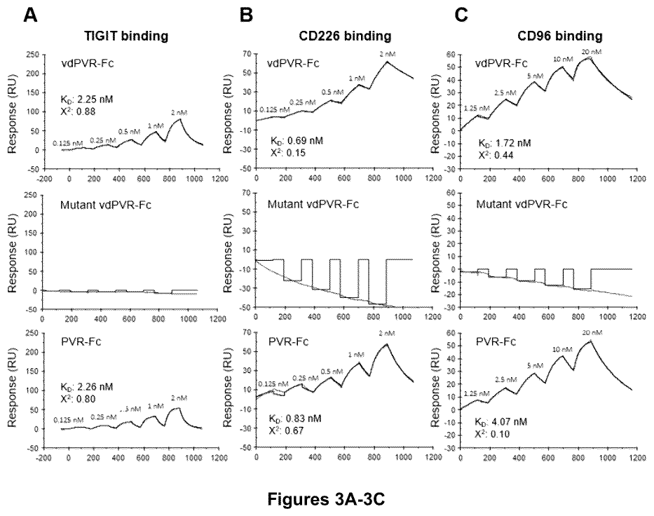

The binding of vdPVR-Fc and mutant vdPVR-Fc to recombinant mouse TIGIT, CD226 and CD96 was assessed using surface plasmon resonance (SPR). vdPVR-Fc was shown to bind to TIGIT-Fc-his, CD96-his, and CD226-his with similar nanomolar affinities (Kd values of 2.25 nM, 1.72 nM and 0.69 nM respectively) (FIG. 3A, 3B, 3C; Table 1). These results were compared to binding constants obtained using full length PVR-Fc to determine whether the modifications to the ECD of PVR impacted its ability to bind to its cognate receptors. Full length PVR-Fc bound to all three cognate receptors with similar low nanomolar affinities (TIGIT-Fc, Kd 2.26 nM; CD96-his, Kd 4.07 nM, CD226-his, Kd 0.83 nM) (FIG. 3A, 3B, 3C; Table 1) as was obtained for vdPVR-Fc. In addition, binding of the PCR (IgV) Fc construct to human TIGIT was retained and the dissociation constant was determined to be in the low nano-molar range (data not shown). The nanomolar affinities and function activity (see below) observed herein for the truncated IgV-Fc construct suggests that this domain alone encodes for the functional features of PVR. Finally, whether or not the mutations introduced in the IgV domain of PVR nullified its ability to bind to its cognate receptors was assessed by comparing the affinities obtained to those of vdPVR-Fc or the full-length mouse PVR ECD-Fc. As expected, the negative control, mutant vdPVR-Fc, did not bind to either mouse TIGIT-Fc, CD226-his or CD96-his as indicated by the lack of increase in response units from the SPR sensorgrams (FIG. 3A, 3B, 3C).

TABLE 1
Summary of vdPVR-Fc and PVR Fc binding kinetic
parameters calculated from SPR sensorgrams

| Interaction | k (1/Ms) | k (1/s) | K (M) | Chi² |
|---|---|---|---|---|
| vdPVR-Fc and | 2.92 × 10⁷ | 6.56 × 10² | 2.25 × 10⁻⁹ | 0.88 |
| mTIGIT | | | | |
| vdPVR-Fc and | 2.67 × 10⁶ | 1.84 × 10⁻³ | 6.90 × 10⁻¹⁰ | 0.15 |
| mCD226 | | | | |
| vdPVR-Fc and | 3.52 × 10⁶ | 2.86 × 10⁻³ | 1.72 × 10⁻⁹ | 0.44 |
| mCD96 | | | | |
| PVR-Fc and | 2.25 × 10⁷ | 6.46 × 10² | 2.26 × 10⁻⁹ | 0.80 |
| mTIGIT | | | | |
| PVR-Fc and | 3.20 × 10⁶ | 6.51 × 10⁻³ | 8.30 × 10⁻¹⁰ | 0.67 |
| mCD226 | | | | |
| PVR-Fc and | 1.17 × 10⁶ | 4.55 × 10⁻³ | 4.07 × 10⁻⁹ | 0.10 |
| mCD96 | | | | |

### References Cited

5. Joller N, et al. Cutting Edge: TIGIT Has T Cell-Intrinsic Inhibitory Functions. The Journal of Immunology 2011;186(3):1338-1342.
6. Stanietsky N, et al. Mouse TIGIT inhibits NK-cell cytotoxicity upon interaction with PVR. Eur J Immunol 2013;43(8):2138.
7. Chan C J, et al. The receptors CD96 and CD226 oppose each other in the regulation of natural killer cell functions. Nat Immunol 2014;15(5):431-438.
8. Tahara-Hanaoka S, et al. Functional characterization of DNAM-1 (CD226) interaction with its ligands PVR (CD155) and nectin-2 (PRR-2/CD112). Int Immunol 2004;16(4):533-538.
9. Johnston R J, et al. The immunoreceptor TIGIT regulates antitumor and antiviral CD8(+) T cell effector function. Cancer Cell 2014;26(6):923-937.
10. Yu X, et al. The surface protein TIGIT suppresses T cell activation by promoting the generation of mature immunoregulatory dendritic cells. Nat Immunol 2009;10(1):48-57.

### Example 3. Assessment of vdPVR-Fc Binding to TIGIT on Murine T Cells Methods

vdPVR-Fc and mutant vdPVR-Fc were synthesized as described in Example 1.

Cell binding to mouse T cells: Binding of vdPVR-Fc and mutant vdPVR-Fc to primary murine CD4⁺ and CD8⁺ T-cells were assessed using flow cytometry. Mouse splenocytes were treated with red blood cell lysis buffer and cultured with about 2 μg/mL Concanavalin A (C5275; MilliporeSigma) for 2 days. Murine splenocytes were harvested, had their Fc receptors blocked with Mouse TruStain FcX (101319; BioLegend) and were incubated with about 2.5 μg of vdPVR-Fc or mutant vdPVR-Fc for about 1 h at about 4° C. Cells were then washed and PE anti-human IgG Fc (409304; Biolegend) was added to detect vdPVR-Fc and mutant PVR-Fc binding. Additionally, cells were simultaneously stained with a cocktail of Alexa Flour 700 anti-CD3 (100216; BioLegend), APC anti-CD8 (100711; BioLegend), and PE/Cy7 anti-CD4 (100422;BioLegend) for about 30 minutes at about 4° C. Cells were subsequently washed and analysed by flow cytometry (BD LSR II, Becton Dickinson; Center for Scanning Microscopy and Flow Cytometry; Sunnybrook Research Institute) with DAPI (D1306; Thermo Fisher Scientific) used to exclude dead cells. To confirm the specificity of vdPVR-Fc to TIGIT, competition cell binding experiments were performed with mouse anti-TIGIT antibody (clone 1B4; Absolute Antibody). Mouse splenocytes were incubated with vdPVR-Fc as described above. Cells were then washed and about a 2-molar excess anti-TIGIT antibody was added along with the other surface markers listed above for about 30 minutes at about 4° C. Flow cytometry analysis was performed using FlowJo version 10.6. (BD Life Sciences).

### Results/Discussion

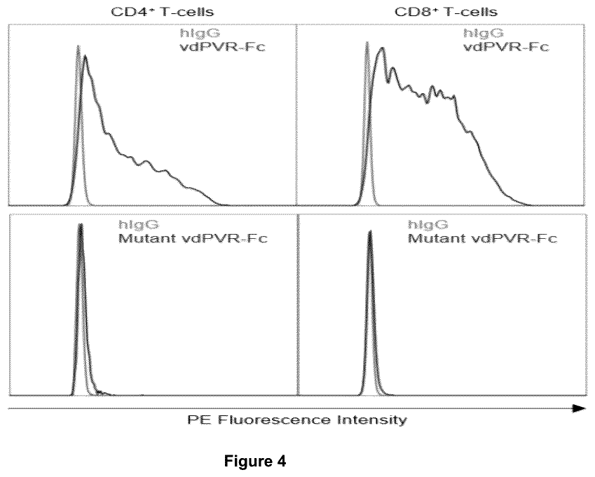

To determine if vdPVR-Fc can bind to the surface of intact cells, its ability to bind to the surface of stimulated T-cells in a mixed splenocyte culture was assessed. Herein, PE-conjugated anti human IgG1-Fc was used to detect vdPVR-Fc binding to murine T-cells by flow cytometry. As shown in FIG. 4, vdPVR-Fc bound to both CD4⁺ and CD8⁺ T-cell subsets as evidenced by the large shift in the PE signal intensity relative to the human IgG1 isotype (top graphs; FIG. 4). In contrast, mutant vdPVR-Fc did not bind to either T-cell subset (bottom graphs; FIG. 4).

### Example 4. Assessment of vdPVR Suppression of Murine T-Cell Activity Ex Vivo Methods

vdPVR-Fc and mutant vdPVR-Fc were synthesized as described in Example 1.

Splenocyte proliferation and cytokine production assay: Mouse splenocytes were treated with red blood cell lysis buffer, stained with about 5 M CFSE (C34554; Thermo Fisher Scientific), and cultured in RPMI-1640 media (Wisent Bio Products) supplemented with about 10% FBS, penicillin (about 100 U/mL), HEPES (about 20 mM) and 2-mercaptoethanol (about 0.05 mM). Cells were plated in round bottom 96-well plates at about 3×10⁵ cells per well and stimulated with Dynabeads™ Mouse T-Activator CD3/CD28 (11452D; Thermo Fisher Scientific). Proteins including vdPVR-Fc and mutant vdPVR-Fc were added at about 10 μg/mL. After 3 days in culture, cells were harvested, Fc receptors on cells were blocked with Mouse TruStain FcX (101319; BioLegend) and cells were stained with a cocktail of Alexa Flour 700 anti-CD3 (100216; BioLegend), APC anti-CD8 (100711; BioLegend), and PE/Cy7 anti-CD4 (100422; BioLegend). T-cell proliferation as defined by the percentage of CFSE signal distributed within dividing T-cells populations was then measured by flow cytometry. IL-2 and IFNγ secretion levels in culture were measured after 3 days using the appropriate ELISA MAX™ Deluxe Set Mouse kits (431004 and 430801; Biolegend). TIGIT, CD226 and CD96 expression on T-cells were assessed under these culture conditions after stimulation with Dynabeads ™ Mouse T-Activator CD3/CD28. Cells were stained with PE anti-TIGIT (142103; Biolegend), PerCP/Cyanine5.5 anti-CD226 (128813; Biolegend), APC anti-CD96 (131711; Biolegend) and the appropriate isotype control. Flow cytometry analysis was performed using FlowJo version 10.6. (BD Life Sciences).

Cytotoxicity assay: Cytotoxicity assays were performed as previously described (31, 32). In brief, about 100,000 murine splenocytes were stimulated with Dynabeads™ Mouse T-Activator CD3/CD28 and cultured in the presence or absence of about 10 μg/mL of either vdPVR-Fc, mutant vdPVR-Fc, human IgG1 isotype control (BioXCell) or anti-CTLA4 (Yervoy; Bristol-Myers Squibb). Following about a 4-h incubation period at about 37° C., supernatants were collected, and cell cytotoxicity assayed using a lactate dehydrogenase kit (ab65393; Abcam). Percent cytotoxicity was calculated with the following formula: cytotoxicity (%)=((Test Sample−Low Control)/(High Control−Low Control))×100.

### Results/Discussion

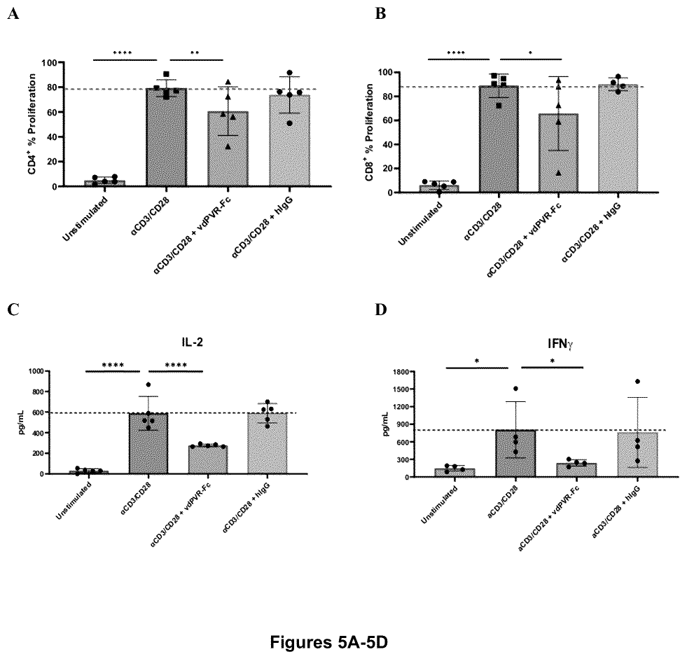

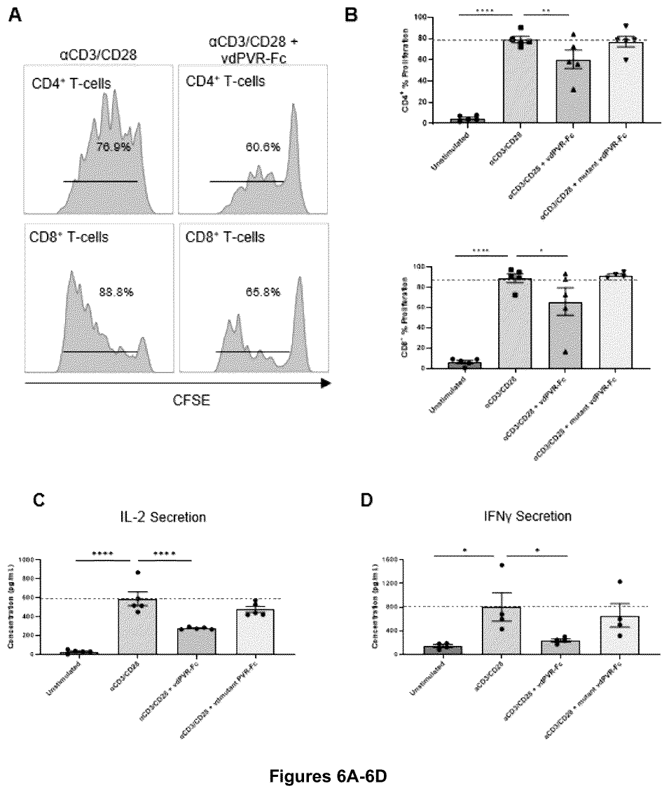

The functional ability of vdPVR-Fc to suppress T-cell activity ex-vivo was tested using anti-CD3/CD28-stimulated murine splenocytes in the presence or absence of (i) vdPVR-Fc or hIgG (FIG. 5A, 5B) and (ii) vdPVR-Fc or mutant vdPVR-Fc (FIG. 6A, 6B). T-cell proliferation responses as well as IL-2 and IFNγ cytokine secretion levels were also assessed for each of (i) (FIG. 5C and 5D) and (ii) (FIG. 6B, 6C, 6D) described above. CFSE histograms of T-cells indicate that the addition of soluble vdPVR-Fc was able to significantly inhibit the proliferation of anti CD3/CD28 stimulated CD4⁺ T-cells (from 76.9% down to 60.6% proliferation; p=0.0062) and CD8⁺ T-cells (from 88.8% down to 65.8% proliferation; p=0.0390; see FIG. 6A) as compared to anti CD3/CD28 stimulation alone (FIGS. 5A, 5B and 6A and 6B).

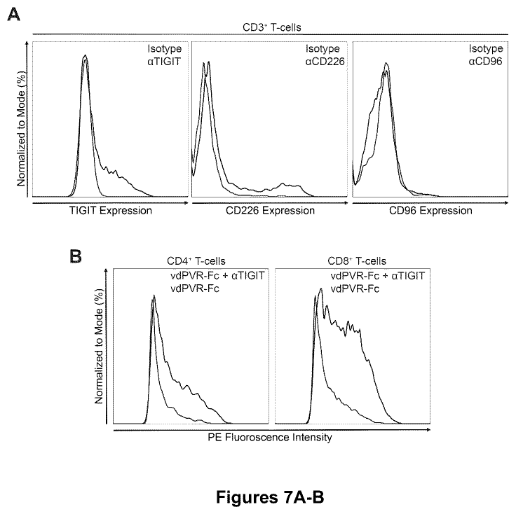

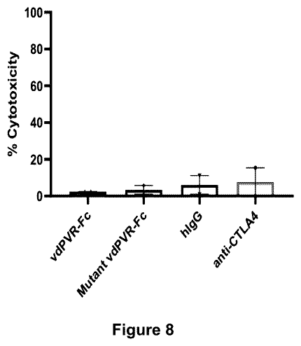

hIgG nor Mutant vdPVR-Fc showed significant changes in either CD4⁺ or CD8⁺ T-cell proliferation compared to anti CD3/CD28 stimulation alone (FIGS. 5A and 5B and FIGS. 6A and 6B). Furthermore, vdPVR-Fc significantly reduced the level of both IL-2 (p<0.0001) and IFNγ (p=0.0459) secretion in the splenocyte culture as compared to anti-CD3/CD28 alone (FIGS. 5C and 5D and FIGS. 6C and 6D). As expected, both hIgG (FIGS. 5C, 5D) and mutant vdPVR-Fc (FIGS. 6C and 6D) had negligible effects on both IL-2 and IFNγ production. Despite CD226 being expressed on the surface of the T-cells (FIG. 7A), a net immunosuppressive effect was observed. To verify that vdPVR-Fc is binding to TIGIT and/or CD96, explaining this suppressive effect, TIGIT expression on the surface of T-cells stimulated ex-vivo was verified (FIG. 7A). The expression of CD96 was unable to be detected in this condition indicating the suppression is likely mediated by TIGIT. These results indicate that the observed effects are not likely due to Fc-mediated toxicity as confirmed by assaying for the presence of lactate dehydrogenase (LDH) in the culture supernatant (FIG. 8). None of the compounds tested were identified as being cytotoxic under these culture conditions.

To further assess the specificity of vdPVR-Fc for TIGIT, an anti-murine TIGIT antibody was used to compete for the binding of vdPVR-Fc on maximally stimulated T-cells (to ensure TIGIT expression). Specifically, the addition of about a 2-molar excess of an anti-TIGIT antibody, known to block the PVR/TIGIT interaction (11), was able to displace vdPVR-Fc binding to the surface of both T-cell subsets (FIG. 7B).

### References Cited

11. Dixon K O, et al. Functional Anti-TIGIT Antibodies Regulate Development of Autoimmunity and Antitumor Immunity. The Journal of Immunology 2018;200(8):3000-3007.
31. Broussas M, Broyer L, Goetsch L. Evaluation of antibody-dependent cell cytotoxicity using lactate dehydrogenase (LDH) measurement. Methods Mol Biol 2013;988:305-317.
32. Ma Y H V, et al. Agonistic nanobodies and antibodies to human VISTA. MAbs 2021;13(1). doi:10.1080/19420862.2021.2003281

### Example 5 Clinical Application—Study in Murine Model of Colitis

T cell immunoreceptor with Ig and ITIM domains (TIGIT) is an immune checkpoint receptor that is reported to suppress T-cell and NK cell activity. PVR (poliovirus receptor) functions as a ligand and signals through TIGIT to induce suppression of T-cell and NK-cell responses. TIGIT inhibitors have been developed to restore immune functions in the context of cancer immunotherapy. While engagement of the TIGIT inhibitory pathway has been previously reported to reduce the severity of EAE and delay the development of systemic lupus erythematosus (11, 12), the therapeutic potential of activating these pathways to treat other inflammatory disorders is relatively unknown. To test this potential, a minimal murine PVR-Fc fusion construct comprising the IgV domain of PVR (vdPVR-Fc) was synthesized and its effect on inflammatory responses in a murine model of colitis was assessed.

### Methods

vdPVR-Fc was synthesized as described in Example 1. For the murine studies, female C57BL/6 mice, about 8 to about 12 weeks of age (Charles River Laboratories) were housed in a pathogen-free environment at the Sunnybrook Research Institute (SRI) Comparative Research facility. All protocols (AUP 658 and 710) were approved by the SRI Comparative Research Animal Care Committee, accredited by the Canadian Council of Animal Care.

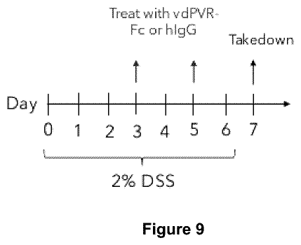

The anti-inflammatory activity of vdPVR-Fc was assessed in vivo in a murine model of colitis. Colitis was induced by adding about 2% (w/v) dextran sulfate sodium (DSS) in the drinking water of female C57BL/6 mice. The induction of colitis and treatment protocol used is shown in FIG. 9. Briefly, mice receiving DSS drinking water for 7 days were intraperitonially treated on Days 3 and 5 with about 80 μg of either vdPVR-Fc or of an isotype control hIgG1. The severity of DSS-induced colitis in mice was evaluated by monitoring body weight, stool consistency and rectal bleeding daily. Their disease activity index (DAI) was calculated by summing the score of weight loss, stool consistency and rectal bleeding using the scoring criteria shown in Table 2. On day 7, mice were sacrificed, and their colons lengths were measured.

TABLE 2
Scoring of Disease Activity Index (DAI)

| | Body weight | Stool | Rectal |
|---|---|---|---|
| Score | decrease (%) | consistency | bleeding |
| 0 | <1 | Normal | Normal |
| 1 | 1-5 | | |
| 2 | 5-10 | Loose stools | |
| 3 | 10-20 | | |
| 4 | >20 | Diarrhea | Gross Bleeding |

### Results/Discussion

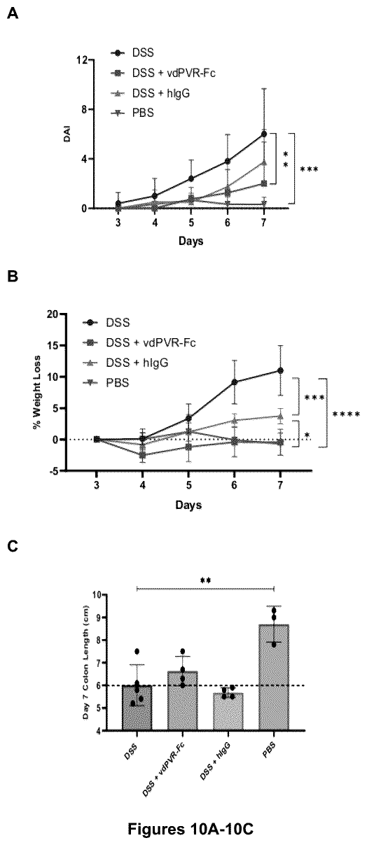

As shown in FIGS. 10A-C, treatment with vdPVR-Fc significantly reduced percent weight loss (p<0.0001) and overall disease activity index scores) as compared to mice that received DSS alone (DAI 2 vs 6 p 0.0061) (FIG. 10A-B). Furthermore, the average colon length (which is expected to diminish upon DSS treatment) was longer in the vdPVR-Fc-treated group as compared to mice treated with an isotype-matched hIgG and mice receiving DSS only, although it was not statistically significant (FIG. 10C).

### References Cited

11. Dixon K O, et al. Functional Anti-TIGIT Antibodies Regulate Development of Autoimmunity and Antitumor Immunity. The Journal of Immunology 2018;200(8):3000-3007.
12. Mao L, et al. TIGIT signalling pathway negatively regulates CD4+ T-cell responses in systemic lupus erythematosus. Immunology 2017;151(3):280-290.

### Example 6—Clinical Application—Study in Murine Model of Psoriasis

PVR (poliovirus receptor) functions as a ligand that signals through TIGIT and CD96 to induce suppression of T-cell and NK-cell responses. Alternatively, PVR binds to CD226, resulting in a co-stimulatory signal. To date, TIGIT antibody antagonists have been developed to restore immune functions and allow PVR to signal though CD226 in the context of cancer immunotherapy. Agonizing either of these pathways with the PVR extracellular domain may represent a therapeutic strategy for either immunosuppression or activation. To test this, a minimal murine PVR-Fc fusion construct comprising the IgV domain of PVR (vdPVR-Fc) was synthesized and its effect on inflammatory responses in a murine model of psoriasis was assessed.

### Methods

vdPVR-Fc and mutant vdPVR-Fc were synthesized as described in Example 1. For the murine studies, female C57BL/6 mice, about 8 to about 12 weeks of age (Charles River Laboratories) were housed in a pathogen-free environment at the Sunnybrook Research Institute (SRI) Comparative Research facility. All protocols (AUP 658 and 710) were approved by the SRI Comparative Research Animal Care Committee, accredited by the Canadian Council of Animal Care. Results from animal studies are reported in accordance with the ARRIVE guidelines (31).

IMQ Induced Psoriasis Model: Female C57BL/6 mice (8-week-old; Charles River Laboratories) received daily topical application on their shaved back of about 62.5 mg of about 5% (w/v) IMQ cream (Aldara; Valeant Pharmaceuticals) or petroleum jelly (Vaseline®). Groups of treated mice (n=13) received intraperitoneal injections every other day starting from day 0 with either vdPVR-Fc (about 100 μg), mutant vdPVR-Fc (about 100 μg) or PBS (about 200 μL). A subset of mice in each group were sacrificed on Day 3 for skin histology and digestion while the remaining mice were treated and scored until Day 5. The severity of the psoriasis-like skin conditions: erythema, scaling, and thickness on a scale ranging from 0 to 4 (no symptoms, 0; mild, 1; moderate, 2; severe, 3; very severe, 4) was recorded daily in each mouse cohort. Scoring was done by 3 independent scorers who were blinded as to the treatment given. The total score was obtained by calculating the sum of the 3 index scores.

Skin Histology: Mice (n=9) were sacrificed on day 3 and had their skin harvested for histological analysis and flow cytometry analysis. Murine back skin sections were digested to assess immune cell populations as previously described. Briefly, precut skin sections were digested with Collegenase P (11213857001; Sigma-Aldrich), DNAse I (9003-98-9; Worthington) and Dispase II (D4693-1G; Sigma-Aldrich) in DMEM (Wisent Bio Products) for 1 hour in 37° C. Cells were then filtered through 40 μm cell strainers before being stained for flow cytometry. Next, Fc receptors on cells were blocked with Mouse TruStain FcX (101319; BioLegend) and stained with cocktail combinations of: APC/Cy7 anti-CD45 (103116; Biolegend), PE/Cy5 anti-CD3 (100310; BioLegend), APC anti-CD8, PE/Cy7 anti-CD4, Alexa Fluora 700 anti-CD11b (101222; Biolegend), FITC anti-NKp46 (137605; Biolegend), PE anti-TIGIT and PerCP Cy5.5 anti-CD226, FITC anti-F4/80 (123108; Biolegend), APC anti-Ly6C (128015; Biolegend), Pe/Cy5 anti-CD11c (117316; Biolegend).

In addition, samples were then sectioned and stained with H&E for assessing epidermal thickness. Histology section images were captured, and epidermal thickness was measured using the S-EYE 1.4 software. The epidermal thickness was quantified by measuring thickness at three distinct locations on each tissue section.

### Results/Discussion

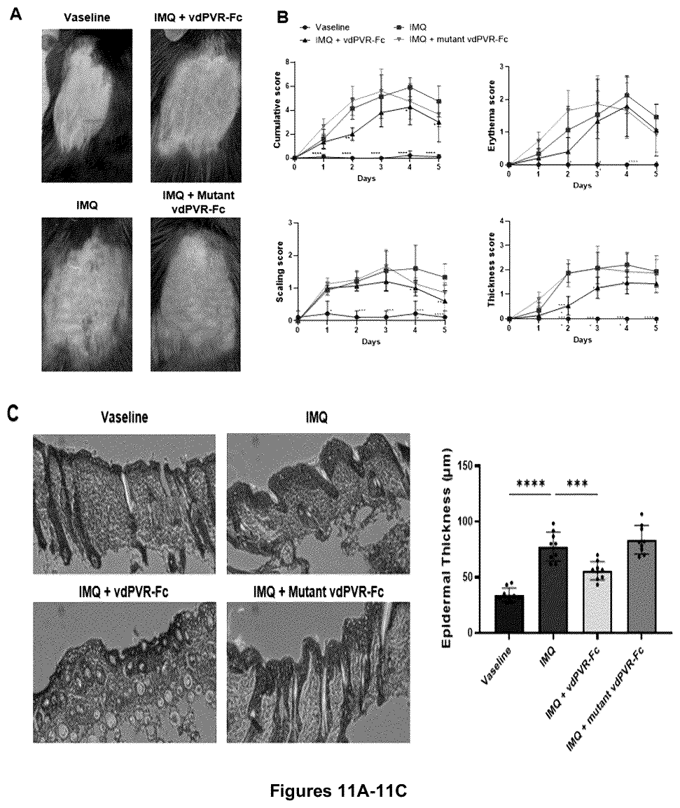

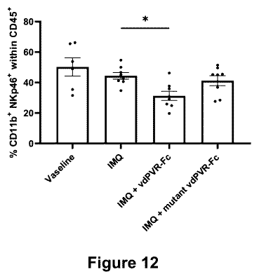

In humans, TIGIT expression is correlated with psoriasis severity (15, 16). Furthermore, CD4⁺ T-cells isolated from patients treated with human PVR-Fc ex-vivo show significant reduction in proliferation and pro-inflammatory cytokine production (15). Despite this, PVR-Fc has not been tested in the context of an in-vivo mouse model of psoriasis, and thus, the effect of treating mice displaying Imiquimod (IMQ)-induced psoriatic lesions with vdPVR-Fc was evaluated. As shown in FIG. 11A, there is a marked improvement in the disease severity early in the study in the mice treated intraperitoneally with vdPVR-Fc as compared to IMQ alone or the mutant vdPVR-Fc. Daily quantification of the symptom severity (Erythema, Scaling and Thickness) (FIG. 11B) indicated a delayed onset of psoriatic symptoms and minimized peak severity (Day 3-4). The delayed onset of disease was particularly evident based on the reduced thickness and scaling of the epidermis observed in vdPVR-Fc-treated mice, as compared to mice receiving IMQ alone or for IMQ-treated mice given mutant vdPVR-Fc. These results were further supported by the subsequent histological assessment of skin sections (FIG. 11C); mice treated with vdPVR-Fc displayed significantly lower epidermal thickness, comparable to Vaseline (vehicle)-treated mice, relative to mice given IMQ alone (p=0.0004) or treated with mutant vdPVR-Fc. These results may be attributed, at least in part, to a significant reduction in CD11b⁺ NKp46⁺ NK cells (p=0.0304) within the skin (FIG. 12). There were no notable differences in any of the other cell populations examined at peak severity (CD11b⁺ F4/80⁺ macrophages, CD11b⁺ CD11c⁺ dendritic cells, CD11b⁺ Ly6C⁺⁺ monocytes, CD4⁺ T-cells and CD8⁺ T-cells (data not shown). Based on these results, vdPVR-Fc is able to reduce the proliferation of T-cells and secretion of pro-inflammatory cytokines ex-vivo, as well as attenuate psoriasiform-like symptoms in an in-vivo mouse model of psoriasis.

### Reference Cited

15. Wang F F, et al. TIGIT expression levels on CD4+ T cells are correlated with disease severity in patients with psoriasis. Clin Exp Dermatol 2018;43(6):675-682.
16. Jacobs M E, et al. DNAM1 and TIGIT balance the T cell response, with low T cell TIGIT expression corresponding to inflammation in psoriatic disease. Immunotherapy Advances 2021;1(1). doi:10.1093/IMMADV/LTAA004
31. du Sert, N. P. et al. Reporting animal research: Explanation and elaboration for the ARRIVE guidelines 2.0. PLoS Biol 18, e3000411 (2020).
34. Lou, F., Sun, Y. & Wang, H. Protocol for Flow Cytometric Detection of Immune Cell Infiltration in the Epidermis and Dermis of a Psoriasis Mouse Model. STAR Protoc 1, (2020).

### Overall Discussion

The PVR/TIGIT/CD226/CD96 signalling axis may represent a promising therapeutic target for both cancer immunotherapy and the treatment of autoimmune diseases. PVR binds to TIGIT and CD96 to suppress T-cell and NK cell functions (5-7). In contrast, PVR engagement of CD226 results in co-stimulatory signalling (8). Accordingly, CD226 agonism in combination with appropriate antagonistic anti-TIGIT and/or anti-CD96 antibodies may represent an attractive therapeutic strategy in the context of cancer immunotherapy. To date, attempts to modulate these immunoregulatory pathways have been centered around TIGIT antagonism and/or CD226 agonism in the context of cancer immunotherapy utilizing antibodies with mixed results in clinical trials (17, 18). The use of natural ligands to agonize immunoregulatory pathways is largely underscored partly due to receptor heterogeneity (i.e. the ligand binds to multiple receptors with opposing outcomes) (19). While receptor heterogeneity holds true for PVR, the interplay between the receptors i.e. TIGIT blocking CD226 mediated co-stimulation, can allow for the development a soluble PVR form as a dual functioning therapeutic with potential utilization in both cancer and auto-immune therapy.

Described and exemplified herein, is a modified version of PVR-Fc incorporating its smallest functional IgV domain (vdPVR-Fc). This smaller biologic can offer advantages over the classic full-length PVR-Fc. For example, Krippendorff et al. reported an inversed relationship between the molecular weight of proteins and their tissue biodistribution (20). In the present study, the reduction of PVR to only its IgV domain, reduces its mass relative to the full length PVR ECD-Fc by ˜70 kDa. This attribute can favor tissue penetration and potentially enhance their therapeutic efficiency. Furthermore, the shortening of the construct can improve production yield (data not shown). The IgC domains of Ig super-family of proteins are typically important for oligomerization and molecular interactions (21, 22) thus removing these domain(s) can render the protein unstable and reduce its binding affinity (13, 23). In this case, removing the two IgC domains from PVR did not impact its ability to bind to its cognate receptors displaying Kd values within ˜2 nM of each other, comparable to values obtained for the full-length ECD version.

Of note, there is conflicting data surrounding the expected affinity of PVR for each of its cognate receptors. In the first instance, two studies reported that the affinity of PVR for TIGIT is considerably higher than its binding to CD226 (10, 24). Yu et al. originally reported a 100-fold difference in affinity when comparing PVR binding to TIGIT as compared to CD226 based on radioligand binding assays on a cell line expressing these receptors (10). In contrast, Stanietsky et al. reported similar affinities for PVR toward TIGIT and CD226, albeit in the micromolar range (6). Complementary to the results presented herein, Okumura et al. recently reported that full length PVR is capable of binding to all three of its cognate receptors in the nanomolar affinity range (25). The differences obtained across multiple studies are likely a reflection of differences in methods used to assess binding affinity (cell binding assay (10) as compared to SPR (6) (Table 1) or Biolayer Interferometry systems) (24, 25). The discrepancy in reported K_{D}s could also be due to differences in the functional format of the cognate receptors and/or PVR used (use of recombinant pure proteins versus cell surface-expressed receptors, glycosylation patterns and oligomeric status). Nevertheless, the results presented herein show that it is unlikely that PVR favors binding to TIGIT over the other receptors and the signaling outcome may vary based on the interplay (signaling/crosstalk) and/or expression levels, of the cognate receptors on T-cells and NK-cells. As expected, no binding to TIGIT, CD96 and CD226 was observed for the inactive version of vdPVR-Fc incorporating two mutations Q63R and F128R (FIG. 3B). These mutations inserted in the binding motifs of PVR have previously been shown to abolish PVR binding to TIGIT and CD226 respectively (13, 14). Overall, the SPR data indicates that the PVR ECD (IgV-IgC-IgC) can be truncated to its IgV domain without a loss of binding affinities to its three cognate receptors.

From a functional perspective, T-cells are known to upregulate TIGIT upon anti-CD3/CD28 co-stimulation (5, 26). The expression of TIGIT was verified on the surface of CD4⁺ and CD8⁺ T-cells post stimulation with anti-CD3/CD28 coated beads. As expected, a net suppressive effect was observed with the addition of vdPVR-Fc based on the interplay between TIGIT and CD226, despite the co-expression of CD226 on these cells. This dampening effect was evidenced by a significant decrease in the proliferation of both CD4⁺ and CD8⁺ T-cells and a decrease in the levels of secreted IL-2 and IFNγ; known cytokine levels impacted by TIGIT agonism (26). These findings, in combination with the lack of observed CD96 expression and the ability of vdPVR-Fc to successfully compete for TIGIT, suggest that the overall inhibitory effect observed in-vivo is TIGIT-mediated.

It was also tested if vdPVR-Fc displayed immunosuppressive properties in an in-vivo mouse model of plaque-like psoriasis. In this model, observable symptoms of skin inflammation are induced by the topical application of imiquimod (IMQ) cream, which activates immune responses as a TLR7 and TLR8 agonist. Full length PVR-Fc has previously shown therapeutic potential evidenced by the delay in the onset of SLE in mice (12). However, pre-clinical work assessing the effect of in vivo administrated PVR construct on the development of psoriasis has not been attempted. One study by Fang et al. demonstrated that TIGIT expression on T cells of psoriasis patients was a predictive indicator of disease severity (15). Additionally, T-cells isolated from psoriasis patients, treated with recombinant PVR ex-vivo, were similarly suppressed as evidenced by a decrease in CD4⁺ T-cell proliferation and reduction in pro-inflammatory cytokine output (15). It remains unclear whether this immunosuppressive effect is mediated by TIGIT or CD96. As presented herein, vdPVR-Fc was demonstrated to delay the onset of psoriatic symptoms and minimized peak severity as compared to IMQ alone or in mice treated with mutant vdPVR-Fc. Based on these results, the effect of vdPVR is elicited early in the model, and while the peak severity of symptoms is reduced, the significance of the protective effect is lost. While TIGIT may be an indicator of disease severity in psoriasis, the results presented herein cannot rule out that the early protective effect of vdPVR could, in part, be mediated though CD96 and that the action of vdPVR-Fc may not be fully restricted to T-cells alone. Indeed, the results from early (Day 3) assessment of immune cells within the skin indicate that vdPVR-Fc treatment results in a significant reduction in the amount of NK cells present. It is known that PVR binds to TIGIT and CD96 on the surface of NK-cells (6, 7). Furthermore, evidence is emerging supporting the involvement of NK-cells in the early development of psoriasis (27). Likewise, and without being bound by theory, it cannot be ruled out that the early protection elicited by vdPVR-Fc was lost due to a switch to CD226 signaling.

More studies are required to determine the exact interplay between various immune cell types and the expression profiles of the PVR cognate receptors within this model before any definitive conclusions can be drawn on the mechanism of action of vdPVR-Fc.

The results presented herein suggest that vdPVR-Fc could be an immune co-stimulatory molecule. A recent study by Fourcade et al. demonstrated that CD226 co-stimulation using PVR-Fc synergistically with TIGIT blockade suppressed melanoma patient derived Tregs ex vivo (28). Furthermore, human cancers are known to differentially express PVR thus modulating the availability of this ligand (29). Collectively, these studies may suggest that one of the factors limiting the success of TIGIT antagonism in the treatment of cancer could be due to the lack of synergistic CD226 co-stimulation. Investigating the potential dual-functionality of vdPVR-Fc as a co-stimulatory reagent in the presence of appropriate anti-TIGIT and anti-CD96 antagonists may be of interest.

Thus, as described and exemplified herein, the PVR-Fc fusion construct comprising a truncated functional IgV domain (vdPVR-Fc), was able to bind to TIGIT, CD226 and CD96 with similar low nanomolar affinities, when compared to PVR-Fc containing the full-length extracellular domain. In addition, vdPVR-Fc displays immunosuppressive activities in the context of inhibiting T-cell (e.g. CD4⁺ and/or CD8⁺ T cells expressing TIGIT) proliferation and cytokine production ex-vivo and can attenuate the development of inflammatory symptoms associated with colitis (e.g. weight loss and/or disease severity) and psoriasis (e.g. erythema, scaling and/or thickness). Thus, the minimal IgV extracellular domain of PVR may be sufficient to dampen T-cell mediated immune responses associated with the expression of TIGIT. Collectively, the results provided herein suggest that activating TIGIT with the IgV extracellular domain of PVR can be useful for treating inflammatory disorders.

### References Cited

5. Joller N, et al. Cutting Edge: TIGIT Has T Cell-Intrinsic Inhibitory Functions. The Journal of Immunology 2011; 186(3):1338-1342.
6. Stanietsky N, et al. Mouse TIGIT inhibits NK-cell cytotoxicity upon interaction with PVR. Eur J Immunol 2013;43(8):2138.
7. Chan C J, et al. The receptors CD96 and CD226 oppose each other in the regulation of natural killer cell functions. Nat Immunol 2014;15(5):431-438.
8. Tahara-Hanaoka S, et al. Functional characterization of DNAM-1 (CD226) interaction with its ligands PVR (CD155) and nectin-2 (PRR-2/CD112). Int Immunol 2004;16(4):533-538.
10. Yu X, et al. The surface protein TIGIT suppresses T cell activation by promoting the generation of mature immunoregulatory dendritic cells. Nat Immunol 2009;10(1):48-57.
12. Mao L, et al. TIGIT signalling pathway negatively regulates CD4+ T-cell responses in systemic lupus erythematosus. Immunology 2017;151(3):280-290.
13. Wang H, et al. Binding mode of the side-by-side two-IgV molecule CD226/DNAM-1 to its ligand CD155/Necl-5. Proc Natl Acad Sci U S A 2019;116(3):988-996.
14. Stengel K F, et al. Structure of TIGIT immunoreceptor bound to poliovirus receptor reveals a cell-cell adhesion and signaling mechanism that requires cis-trans receptor clustering. Proc Natl Acad Sci U S A 2012;109(14):5399-5404.
15. Wang F F, et al. TIGIT expression levels on CD4+ T cells are correlated with disease severity in patients with psoriasis. Clin Exp Dermatol 2018;43(6):675-682.
17. Chiang E Y, Mellman I. TIGIT-CD226-PVR axis: advancing immune checkpoint blockade for cancer immunotherapy. J Immunother Cancer 2022;10(4):e004711.
18. Conner M, et al. Emergence of the CD226 Axis in Cancer Immunotherapy. Front Immunol 2022;0:3117.
19. Paluch C, et al. Immune Checkpoints as Therapeutic Targets in Autoimmunity. Front Immunol 2018;9(OCT):2306.
20. Li Z, et al. Influence of molecular size on tissue distribution of antibody fragments. MAbs 2016;8(1):113-119.
21. Bork P, Holm L, Sander C. The immunoglobulin fold: Structural classification, sequence patterns and common core. J Mol Biol 1994;242(4). doi:10.1006/jmbi.1994.1582
22. Halaby D M, Mornon J P E. The immunoglobulin superfamily: An insight on its tissular, species, and functional diversity. J Mol Evol 1998;46(4). doi:10.1007/PL00006318
23. Chattopadhyay K, et al. Structural basis of inducible costimulator ligand costimulatory function: determination of the cell surface oligomeric state and functional mapping of the receptor binding site of the protein. J Immunol 2006;177(6):3920-3929.
24. Zhang H, et al. Recombinant oncolytic adenovirus expressing a soluble PVR elicits long-term antitumor immune surveillance. Mol Ther Oncolytics 2021;20:12.
25. Okumura G, et al. Tumor-derived soluble CD155 inhibits DNAM-1-mediated antitumor activity of natural killer cells. J Exp Med 2020;217(4). doi:10.1084/JEM.20191290
26. Lozano E, et al. The TIGIT/CD226 axis regulates human T cell function doi:10.4049/jimmunol.1103627
27. Gardiner C M, Dunphy S. NK cells and psoriasis. J Biomed Biotechnol 2011;2011. doi:10.1155/2011/248317
28. Fourcade J, et al. CD226 opposes TIGIT to disrupt Tregs in melanoma. JCI Insight 2018;3(14). doi:10.1172/JCI.INSIGHT.121157
29. Lee B R, et al. Combination of PD-L1 and PVR determines sensitivity to PD-1 blockade. JCI Insight 2020;5(14). doi:10.1172/JCI.INSIGHT.128633

The above disclosure generally describes the present invention. Although specific terms have been employed herein, such terms are intended in a descriptive sense and not for purposes of limitation.

The above disclosure generally describes the present invention. Although specific terms have been employed herein, such terms are intended in a descriptive sense and not for purposes of limitation. All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. A construct comprising a truncated poliovirus receptor (PVR) and a Fc domain.

2. (canceled)

3. The construct of claim 1, wherein the truncated PVR is free of both IgC domains.

4. (canceled)

5. The construct of claim 1, wherein the truncated PVR comprises an immunoglobulin-like variable (IgV) extracellular domain (ECD).

6. The construct of claim 5, wherein the IgV ECD comprises a truncated murine IgV ECD.

7-10. (canceled)

11. The construct of claim 5, wherein the truncated IgV ECD comprises or consists residues 29 to 147 of SEQ ID NO: 1.

12-14. (canceled)

15. The construct of claim 1, wherein the Fc domain is a human IgG1 Fc domain comprising or consisting of a polypeptide having at least 80% sequence identity to SEQ ID NO:3
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPGK
or a fragment thereof; or
wherein the Fc domain is a human IgG1 Fc domain comprising or consisting of a polypeptide having at least 85, 90, 95, 96, 97, 98, or 99% identity to SEQ ID NO:3, or a fragment thereof.

16-19. (canceled)

20. The construct of claim 1, wherein the Fc domain is a human IgG1 Fc domain comprising or consisting of residues 100 to 330 of SEQ ID NO:3.

21-26. (canceled)

27. The construct of claim 5, wherein the truncated IgV ECD is at the N-terminus of the construct and the Fc domain is at the C-terminus of the construct or wherein the Fc domain is at the N-terminus of the construct and the truncated IgV ECD is at the C-terminus of the construct.

28. (canceled)

29. The construct of claim 1, further comprising a linker between the PVR and the Fc domain.

30. The construct of claim 29, wherein the linker comprises or consists of the sequence IEGRMDP (SEQ ID NO:5).

31. The construct of claim 1, further comprising a secretion signal.

32. The construction of claim 31, wherein the secretion signal comprises or consists of the sequence METDTLLLWVLLLWVPGSTG (SEQ ID NO: 6)

33. The construct of claim 1, comprising or consisting of a polypeptide having at least 80% sequence identity to SEQ ID NO:7:
METDTLLLWVLLLWVPGSTGdirvlvpynstgvlggsttlhcsltsnenv
titqitwmkkdsggshalvavfhpkkgpnikepervkflaaqqdlrnasl
aisnlsvedegiyecqiatfprgsrstnawlkVQARPKNIEGRMDPTHTC
PPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCS
VMHEALHNHYTQKSLSLSPGK
or a fragment thereof; or
comprising or consisting of a polypeptide having at least 85, 90, 95, 96, 97, 98, or 99% identity to SEQ ID NO:7, or a fragment thereof.

34. (canceled)

35. The construct of claim 1, comprising or consisting of a polypeptide having SEQ ID NO:7.

36-38. (canceled)

39. The construct of claim 1, wherein, at least one of:
(a) the construct is soluble;
(b) the construct can bind to cognate receptors with similar affinity as compared to a construct comprising a non-truncated PVR;
(c) the construct binds to the cognate receptor at low nanomolar affinities, such as, about 0.1 nM to about 10 nM;
(d) the construct binds to TIGIT with an affinity of about 2.2 nM;
(e) the construct binds to CD96 with an affinity of about 1.7 nM;
(f) the construct binds to CD226 with an affinity of about 0.7 nM;
(g) the construct suppresses immune responses, wherein the immune response comprises a cytokine response or wherein the immune response comprises proliferation of immune cells;
(h) the construct reduces symptoms associated with a inflammatory disease or condition or for treating an inflammatory disease or condition selected from Crohn's disease, ulcerative colitis, multiple sclerosis, asthma, rheumatoid arthritis, or psoriasis; and
(i) delays the onset of psoriasis.

40-58. (canceled)

59. A polynucleotide encoding the construct of claim 1, comprising or consisting of a polynucleotide having at least 80% sequence identity to SEQ ID NO:9:
ATGGAAACCGACACACTGCTGCTGTGGGTGCTGCTTCTTTGGGTGCCCGG
AAGCACCGGCGATATCAGAGTGCTGGTGCCCTACAATTCCACCGGCGTGC
TCGGCGGATCTACCACACTGCACTGTAGCCTGACCAGCAACGAGAACGTG
ACCATCACACAGATCACCTGGATGAAGAAGGACAGCGGCGGCTCTCATGC
TCTGGTGGCCGTGTTTCACCCTAAGAAGGGCCCCAACATCAAAGAACCCG
AGAGAGTGAAGTTCCTGGCCGCTCAGCAGGACCTGAGAAATGCCTCTCTG
GCCATCAGCAACCTGAGCGTGGAAGATGAGGGCATCTACGAGTGCCAGAT
CGCCACCTTTCCTAGAGGCAGCAGAAGCACCAACGCCTGGCTGAAGGTTC
AGGCCAGACCTAAGAACATCGAGGGCAGAATGGACCCCACACACACCTGT
CCTCCATGTCCTGCTCCAGAGCTGCTCGGAGGCCCTTCCGTGTTTCTGTT
CCCTCCAAAGCCTAAGGACACCCTGATGATCAGCAGAACCCCTGAAGTGA
CCTGCGTGGTGGTGGATGTGTCCCACGAGGATCCCGAAGTGAAGTTTAAT
TGGTACGTGGACGGCGTGGAAGTGCACAACGCCAAGACCAAGCCTAGAGA
GGAACAGTACAACAGCACCTACAGAGTGGTGTCCGTGCTGACCGTGCTGC
ACCAGGATTGGCTGAACGGCAAAGAGTACAAGTGCAAGGTGTCCAACAAG
GCCCTGCCTGCTCCTATCGAGAAAACCATCTCCAAGGCCAAGGGCCAGCC
TAGGGAACCCCAGGTTTACACACTGCCTCCAAGCCGGGAAGAGATGACCA
AGAACCAGGTGTCCCTGACCTGCCTGGTCAAGGGCTTCTACCCTAGCGAC
ATTGCCGTGGAATGGGAGAGCAATGGCCAGCCTGAGAACAACTACAAGAC
CACACCTCCTGTGCTGGACAGCGACGGCTCATTCTTCCTGTACAGCAAGC
TGACAGTGGACAAGAGCAGATGGCAGCAGGGCAACGTGTTCAGCTGCAGC
GTGATGCACGAGGCCCTGCACAACCACTACACCCAGAAGTCCCTGAGCCT
GTCTCCTGGCAAGTGATGATGAA
or a fragment thereof; or
comprising or consisting of a polynucleotide having at least 80% sequence identity to SEQ ID NO:10:
AUGGAAACCGACACACUGCUGCUGUGGGUGCUGCUUCUUUGGGUGCCCGG
AAGCACCGGCGAUAUCAGAGUGCUGGUGCCCUACAAUUCCACCGGCGUGC
UCGGCGGAUCUACCACACUGCACUGUAGCCUGACCAGCAACGAGAACGUG
ACCAUCACACAGAUCACCUGGAUGAAGAAGGACAGCGGCGGCUCUCAUGC
UCUGGUGGCCGUGUUUCACCCUAAGAAGGGCCCCAACAUCAAAGAACCCG
AGAGAGUGAAGUUCCUGGCCGCUCAGCAGGACCUGAGAAAUGCCUCUCUG
GCCAUCAGCAACCUGAGCGUGGAAGAUGAGGGCAUCUACGAGUGCCAGAU
CGCCACCUUUCCUAGAGGCAGCAGAAGCACCAACGCCUGGCUGAAGGUUC
AGGCCAGACCUAAGAACAUCGAGGGCAGAAUGGACCCCACACACACCUGU
CCUCCAUGUCCUGCUCCAGAGCUGCUCGGAGGCCCUUCCGUGUUUCUGUU
CCCUCCAAAGCCUAAGGACACCCUGAUGAUCAGCAGAACCCCUGAAGUGA
CCUGCGUGGUGGUGGAUGUGUCCCACGAGGAUCCCGAAGUGAAGUUUAAU
UGGUACGUGGACGGCGUGGAAGUGCACAACGCCAAGACCAAGCCUAGAGA
GGAACAGUACAACAGCACCUACAGAGUGGUGUCCGUGCUGACCGUGCUGC
ACCAGGAUUGGCUGAACGGCAAAGAGUACAAGUGCAAGGUGUCCAACAAG
GCCCUGCCUGCUCCUAUCGAGAAAACCAUCUCCAAGGCCAAGGGCCAGCC
UAGGGAACCCCAGGUUUACACACUGCCUCCAAGCCGGGAAGAGAUGACCA
AGAACCAGGUGUCCCUGACCUGCCUGGUCAAGGGCUUCUACCCUAGCGAC
AUUGCCGUGGAAUGGGAGAGCAAUGGCCAGCCUGAGAACAACUACAAGAC
CACACCUCCUGUGCUGGACAGCGACGGCUCAUUCUUCCUGUACAGCAAGC
UGACAGUGGACAAGAGCAGAUGGCAGCAGGGCAACGUGUUCAGCUGCAGC
GUGAUGCACGAGGCCCUGCACAACCACUACACCCAGAAGUCCCUGAGCCU
GUCUCCUGGCAAGUGAUGAUGAA
or a fragment thereof.

60-63. (canceled)

64. A vector comprising the polynucleotide of claim 59.

65. A host cell comprising the vector of claim 64.

66. A composition comprising the construct of claim 1.

67-95. (canceled)


SEQUENCE LISTING (13 sequences)

SEQ ID NO: 1 - protein, 408 aa, Mus musculus
MAQLARATRSPLSWLLLLFCYALRKAGGDIRVLVPYNSTGVLGGSTTLHCSLTSNENVTITQITWMKKDSGGSHALVAVFHPKKGPNIKEPERVKFLAAQQDLRNASLAISNLSVEDEGIYECQIATFPRGSRSTNAWLKVQARPKNTAEALEPSPTLILQDVAKCISANGHPPGRISWPSNVNGSHREMKEPGSQPGTTTVTSYLSMVPSRQADGKNITCTVEHESLQELDQLLVTLSQPYPPENVSISGYDGNWYVGLTNLTLTCEAHSKPAPDMAGYNWSTNTGDFPNSVKRQGNMLLISTVEDGLNNTVIVCEVTNALGSGQGQVHIIVKEKPENMQQNTRLHLGYIFLIVFVLAVVIIIAALYTIRRCRHGRALQSNPSERENVQYSSVNGDCRLNMEPNSTR

SEQ ID NO: 2 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 3 - protein, 330 aa, Homo sapiens [sequence omitted]

SEQ ID NO: 4 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 7 aa, synthetic construct
IEGRMDP

SEQ ID NO: 6 - protein, 20 aa, synthetic construct
METDTLLLWVLLLWVPGSTG

SEQ ID NO: 7 - protein, 371 aa, synthetic construct
METDTLLLWVLLLWVPGSTGDIRVLVPYNSTGVLGGSTTLHCSLTSNENVTITQITWMKKDSGGSHALVAVFHPKKGPNIKEPERVKFLAAQQDLRNASLAISNLSVEDEGIYECQIATFPRGSRSTNAWLKVQARPKNIEGRMDPTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 365 aa, synthetic construct
METDTLLLWVLLLWVPGSTGPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - DNA, 1123 bp, synthetic construct
  source 1..1123: mol_type = other DNA
atggaaaccgacacactgctgctgtgggtgctgcttctttgggtgcccggaagcaccggcgatatcagagtgctggtgccctacaattccaccggcgtgctcggcggatctaccacactgcactgtagcctgaccagcaacgagaacgtgaccatcacacagatcacctggatgaagaaggacagcggcggctctcatgctctggtggccgtgtttcaccctaagaagggccccaacatcaaagaacccgagagagtgaagttcctggccgctcagcaggacctgagaaatgcctctctggccatcagcaacctgagcgtggaagatgagggcatctacgagtgccagatcgccacctttcctagaggcagcagaagcaccaacgcctggctgaaggttcaggccagacctaagaacatcgagggcagaatggaccccacacacacctgtcctccatgtcctgctccagagctgctcggaggcccttccgtgtttctgttccctccaaagcctaaggacaccctgatgatcagcagaacccctgaagtgacctgcgtggtggtggatgtgtcccacgaggatcccgaagtgaagtttaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagccgggaagagatgaccaagaaccaggtgtccctgacctgcctggtcaagggcttctaccctagcgacattgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaagtgatgatgaa

SEQ ID NO: 10 - RNA, 1123 nt, synthetic construct
  source 1..1123: mol_type = other RNA
atggaaaccgacacactgctgctgtgggtgctgcttctttgggtgcccggaagcaccggcgatatcagagtgctggtgccctacaattccaccggcgtgctcggcggatctaccacactgcactgtagcctgaccagcaacgagaacgtgaccatcacacagatcacctggatgaagaaggacagcggcggctctcatgctctggtggccgtgtttcaccctaagaagggccccaacatcaaagaacccgagagagtgaagttcctggccgctcagcaggacctgagaaatgcctctctggccatcagcaacctgagcgtggaagatgagggcatctacgagtgccagatcgccacctttcctagaggcagcagaagcaccaacgcctggctgaaggttcaggccagacctaagaacatcgagggcagaatggaccccacacacacctgtcctccatgtcctgctccagagctgctcggaggcccttccgtgtttctgttccctccaaagcctaaggacaccctgatgatcagcagaacccctgaagtgacctgcgtggtggtggatgtgtcccacgaggatcccgaagtgaagtttaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagccgggaagagatgaccaagaaccaggtgtccctgacctgcctggtcaagggcttctaccctagcgacattgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaagtgatgatgaa

SEQ ID NO: 11 - DNA, 1104 bp, synthetic construct
  source 1..1104: mol_type = other DNA
atggaaaccgacacactgctgctgtgggtgctgcttctttgggtgcccggatctacaggacctggaacaggcgacgttgtggtgcaggctcctactcaggtgccaggctttctgggcgatagcgtgaccctgccttgctacctgcaagtgcccaacatggaagtgacccacgtgtcccagctgacctgggctagacatggcgagtctggatccatggccgtgttccaccagacacagggccctagctacagcgagagcaagcggctggaatttgtggccgctagactgggagccgagctgagaaatgccagcctgagaatgttcggcctgagagtggaagatgagggcaactacacctgtctgttcgtgacctttccacagggcagcagaagcgtggacatctggctggaatctaagtacggccctccttgtcctagctgccccgctcctgaatttctcggcggaccttccgtgttcctgtttcctccaaagcctaaggacaccctgatgatcagcagaacccctgaagtgacctgcgtggtggtggatgtgtcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggacagcccgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctgggcaagtgatgatga

SEQ ID NO: 12 - RNA, 1104 nt, synthetic construct
  source 1..1104: mol_type = other RNA
atggaaaccgacacactgctgctgtgggtgctgcttctttgggtgcccggatctacaggacctggaacaggcgacgttgtggtgcaggctcctactcaggtgccaggctttctgggcgatagcgtgaccctgccttgctacctgcaagtgcccaacatggaagtgacccacgtgtcccagctgacctgggctagacatggcgagtctggatccatggccgtgttccaccagacacagggccctagctacagcgagagcaagcggctggaatttgtggccgctagactgggagccgagctgagaaatgccagcctgagaatgttcggcctgagagtggaagatgagggcaactacacctgtctgttcgtgacctttccacagggcagcagaagcgtggacatctggctggaatctaagtacggccctccttgtcctagctgccccgctcctgaatttctcggcggaccttccgtgttcctgtttcctccaaagcctaaggacaccctgatgatcagcagaacccctgaagtgacctgcgtggtggtggatgtgtcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggacagcccgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctgggcaagtgatgatga

SEQ ID NO: 13 - protein, 8 aa, synthetic construct
DYKDDDDK